(19) United States
(12) Patent Application Publication
(10) Pub. No.: US 2011/0003884 A1
(43) Pub. Date: Jan. 6, 2011

(54) Viral Vectors and Methods of Use

(76) Inventors: Konstantin V. Pugachev, Natick, MA (US); Qing-sheng Gao, Belmont, MA (US); Alexander A. Rumyantsev, Somerville, MA (US)

Correspondence Address: CLARK & ELBING LLP, 101 FEDERAL STREET, BOSTON, MA, 02110, US

(21) Appl. No.: 12/526,140

(22) PCT Filed: Feb. 11, 2008
(86) PCT No.: PCT/US08/01777

§ 371 Date: Mar. 18, 2010

Related U.S. Application Data
(60) Provisional application No. 60/900,672, filed on Feb. 9, 2007.

(51) Int. Cl. A61K 31/7088; C12N 15/63

(52) U.S. Cl. 514/44 R; 435/320.1

(57) ABSTRACT

This invention relates to viral vectors and methods employing these vectors. The vectors of the invention can be base on flaviviruses, such as chimeric flavi viruses, which may be used to deliver heterologous antigens, such as influenza virus antigens.

### FIELD OF THE INVENTION

This invention relates to viral vectors and methods employing these vectors.

### BACKGROUND OF THE INVENTION

Influenza virus is a major cause of acute respiratory disease worldwide. Yearly outbreaks are responsible for more than 100,000 hospitalizations and 20,000 to 40,000 deaths in the U.S. alone (Brammer et al., MMWR Surveill. Summ. 51:1-10, 2002; Liu et al., Am. J. Public Health 77:712-716, 1987; Simonsen, Vaccine 17(1):S3-10, 1999; Thompson et al., J.A.M.A. 289:179-186, 2003). Approximately 20% of children and 5% of adults worldwide become ill due to influenza annually (Thompson et al., J.A.M.A. 289:179-186, 2003). Historically, three subtypes of influenza A virus circulate in human populations, H1N1, H2N2, and H3N2. Since 1968, H1N1 and H3N2 have circulated almost exclusively (Hilleman, Vaccine 20:3068-3087, 2002; Thompson et al., J.A.M.A. 289:179-186, 2003; Palese et al., J. Clin. Invest. 110:9-13, 2002). Influenza B virus, of which there is only one recognized subtype, also circulates in humans, but generally causes a milder disease than do influenza A viruses (Hilleman, Vaccine 20:3068-3087, 2002; Murphy et al., in Fields Virology, Third Edition, Fields et al. (Eds.), Lippincott-Raven, Philadelphia, 1397-1445, 1996; Nicholson et al., Lancet 362:1733-1745, 2003). Current inactivated vaccines contain three components, based on selected H1N1 and H3N2 influenza A strains and one influenza B strain (Palese et al., J. Clin. Invest. 110:9-13, 2002).

Periodic pandemics, such as the H1N1 pandemic of 1918, can kill millions of people. Influenza experts agree that another influenza pandemic is inevitable and may be imminent (Webby et al., Science 302:1519-1522, 2003). The recent outbreak of H5N1 avian influenza—the largest on record, caused by a highly lethal strain to humans—has the potential (through mutation and/or genetic re-assortment) to become a pandemic strain, with devastating consequences. Another alarming situation arose in 2003 in the Netherlands, where a small but highly pathogenic H7N7 avian influenza outbreak occurred in poultry industry workers. Other subtypes that pose a pandemic threat are H9 and H6 viruses. Although less virulent than the H5 and H7 viruses, both have spread from aquatic birds to poultry during the past 10 years. Further, H9N2 viruses have been detected in pigs and humans (Webby et al., Science 302:1519-1522, 2003). Despite the large amount of attention received by avian viruses in the past few years, still the traditional H1, H2, and H3 subtype viruses continue to represent a concern, because highly virulent strains can emerge due to introduction of new antigenically distant strains. For example, H2 viruses are in the high-risk category, because they were the causative agents of the 1957 “Asian” flu pandemic and continue to circulate in wild and domestic ducks.

The current strategy for prevention and control of influenza disease is yearly vaccination against the virus strains likely to be circulating that year. Most licensed influenza vaccines are produced in embryonated chicken eggs and consist of inactivated whole virions or partially purified virus subunits (“split” vaccines). These vaccines are 70 to 90% efficacious in normal healthy adults (Beyer et al., Vaccine 20:1340-1353, 2002). However, efficacy against disease is poorer in the elderly.

Live, attenuated intranasal vaccines, also manufactured in embryonated eggs, are available in the U.S. and the former Soviet Union (Treanor et al., in New Generation Vaccines, Third Edition, Levine et al. (Eds.), Marcel Dekker, New York, Basel, 537-557, 2004). The U.S. vaccine (Flumist®) is not approved for use in children under 5 or for persons over 55 years of age, the principal target populations for influenza vaccination.

Because the major influenza hemagglutinin and neuraminidase proteins recognized by the immune system are continually changing by mutation and re-assortment, the vaccine composition has to be altered annually to reflect the antigenic characteristics of the then circulating virus strains. Thus, current vaccines must be prepared each year, just before influenza season, and cannot be stockpiled for use in the case of a pandemic. Moreover, the use of embryonated eggs for manufacture is very inefficient. Only 1 to 2 human doses of inactivated vaccine are produced from each egg. A sufficient supply of pathogen-free eggs is a current manufacturing limitation for conventional vaccines. Even during interpandemic periods, 6 months are typically required to produce sufficient quantities of annual influenza vaccines (Gerdil, Vaccine 21:1776-1779, 2003). There are several development efforts underway to manufacture influenza vaccines in cell culture. However, there are also a number of challenges associated with this approach, in particular the use of unapproved cell lines. Whether eggs or cell cultures are used for vaccine production, reverse genetics or genetic re-assortment methods must be employed to convert the new circulating virus strain for which a vaccine is desired into a production strain that replicates to sufficient titer for manufacturing. All of these attributes associated with conventional influenza vaccines are unacceptable in the face of an influenza pandemic.

The development of influenza vaccines based on recombinant hemagglutinin (HA) or HA delivered by adenovirus or alphavirus vectors improves manufacturing efficiency, but does not address the problem of annual genetic drift and the requirement to re-construct the vaccine each year.

In summary, the following challenges with current influenza vaccines are recognized:

1. Low efficacy in the case of poor vaccine and virus strain match; limited age range for live cold-adapted vaccines.

2. Requirement to make new vaccines annually to address antigenic changes in the virus.

3. Low vaccine manufacturing yields.

4. Time for construction of appropriate reassortant viruses for manufacture.

5. Insufficient manufacturing capacity to meet the demands of a pandemic.

6. Biosafety concerns during large-scale manufacture of inactivated pathogenic viruses.

7. Adverse reactions in vaccines allergic to egg products, or due to insufficient attenuation in the case of some live cold-adapted virus vaccines (Treanor et al., in New Generation Vaccines, Third Edition, Levine et al. (Eds.), Marcel Dekker, New York, Basel, 537-557, 2004).

All effective conventional influenza vaccines elicit virus-neutralizing antibodies against HA, which currently represents the immune correlate of protection. However, the antigenicity of HA changes annually. In recent years, other influenza virus proteins have attracted attention as vaccine targets. The M2 protein and, in particular, the ectodomain of M2 (M2e), is highly conserved among influenza A viruses. FIG. 1A provides an alignment of human and avian M2e sequences. Not only is the M2e domain of human influenza viruses conserved among themselves, avian virus M2e sequences are also closely aligned. The highest level of sequence conservation resides in the N-terminal portion of M2e. It is thus noteworthy that it has been shown that the N-terminal 13 amino acids of the M2e peptide (shadowed in the alignment) are primarily responsible for the induction of protective antibodies (Liu et al., FEMS Immunol. Med. Microbiol. 35:141-146, 2003; Liu et al., Immunol. Lett. 93:131-136, 2004; Liu et al., Microbes. Infect. 7:171-177, 2005).

M2e represents the external 23-amino acid portion of M2, a minor surface protein of the virus. While not prominent in influenza virions, M2 is abundantly expressed on the surface of virus-infected cells (Lamb et al., in Fields Virology, Fourth Edition, Knipe (Ed.), Lippincott Williams and Wilkins, Philadelphia, 1043-1126, 2001). However, during normal influenza virus infection, or upon immunization with conventional vaccines, there is very little antibody response to M2 or the M2e determinant. Nevertheless, a non-virus neutralizing monoclonal antibody directed against the M2 protein was shown to be protective in a lethal mouse model of influenza upon passive transfer (Fan et al., Vaccine 22:2993-3003, 2004; Mozdzanowska et al., Vaccine 21:2616-2626, 2003; Treanor et al., J. Virol. 64:1375-1377, 1990).

Antibodies to M2 or M2e do not neutralize the virus but, rather, reduce efficient virus replication sufficiently to protect against symptomatic disease. It is believed that the mechanism of protection elicited by M2 involves NK cell-mediated antibody-dependent cellular cytotoxicity (ADCC). Antibodies against the M2e ectodomain (predominantly of the IgG2a subclass) recognize the epitope displayed on virus-infected cells, which predestines the elimination of infected cells by natural killer (NK) cells (Jegerlehner et al., J. Immunol. 172:5598-5605, 2004). Because the immunity elicited by M2 is not sterilizing, limited virus replication is allowed following infection, which serves to stimulate a broad-spectrum anti-influenza immune response. Theoretically, this could lead to a longer, stronger immunologic memory and better protection from subsequent encounters with the same virus or heterologous strains (Treanor et al., in New Generation Vaccines, Third Edition, Levine et al. (Eds.), Marcel Dekker, New York, Basel, 537-557, 2004).

Walter Fiers and coworkers (Ghent University, Belgium) demonstrated the potential of M2e-based vaccines by genetically fusing the M2e determinant to the hepatitis B virus core protein, which when expressed in bacteria, resulted in M2e presentation on the surface of hepatitis B virus core particles (HBc) (Fiers et al., Virus Res. 103:173-176, 2004; Neirynck et al., Nat. Med. 5:1157-1163, 1999). These HBc-M2e particles were shown to be immunogenic in mice and ferrets, and protective in an influenza virus challenge model in each species.

Another conserved influenza virus domain is the maturation cleavage site of the HA precursor protein, HA₀. Its high level of conservation (Macken et al., in Options for the Control of Influenza (IV), Osterhaus et al. (Eds.), Elsevier Science, Amsterdam, the Netherlands, 103-106, 2001) is due to two functional constraints. First, the sequence must remain a suitable substrate for host proteases releasing the two mature HA subunits, HA₁ and HA₂. Second, the N-terminus of HA₂ contains the fusion peptide that is crucial for infection (Lamb et al., in Fields Virology, Fourth Edition, Knipe (Ed.), Lippincott Williams and Wilkins, Philadelphia, 1043-1126, 2001). The fusion peptide is conserved in both influenza A and B viruses. In a recent report, Bianchi and co-workers (Bianchi et al., J. Virol. 79:7380-7388, 2005) demonstrated that a conjugated HA₀ cleavage peptide of influenza B virus elicited protective immunity in mice against lethal challenge with antigenically distant influenza B virus lineages. A conjugated A/H3/HA₀ peptide also protected immunized mice from influenza B challenge. The strictly conserved Arg at the −1 position (the last HA₁ residue preceding the cleavage point), and the +3 and +9 Phe residues (the 3^{rd} and 9^{th} residues of HA₂) were critical for binding of monoclonal antibodies. Our alignment of the human (H1, H2, H3, and B) HA₀ and all available avian influenza HA₀ sequences resulted in the consensus sequences (the region critical for antibody binding and immunogenicity is shadowed) shown in FIG. 1B.

Various M2e subunit vaccine approaches are being pursued, including peptide conjugates and epitope-displaying particles. However, these approaches require powerful adjuvants to boost the immunogenicity of these weak immunogens. This is particularly critical in the case of M2e (and likely HA₀). Because of the proposed mechanism of protection (ADCC), high levels of specific antibodies are required for efficacy. It is thought that normal serum IgG competes with specific (anti-M2e) IgG for the Fc receptors on NK cells, which are the principal mediators of protection. Thus, alternative approaches to universal pandemic influenza vaccines need to be explored. The above description of the medical significance of influenza, the need for an improved universal influenza vaccine, and the availability of appropriate epitopes/antigens of influenza virus provide one example of an important pathogen for which a new vaccine can be created using approaches described in this invention. Methods described in this invention can be equally applicable to the construction of new/improved vaccines against other pathogens, as described below.

### SUMMARY OF THE INVENTION

The invention provides flavivirus vectors stably expressing one or more heterologous sequences inserted at an intergenic site between envelope (E) and non-structural-1 (NS1) proteins of the flavivirus vector. The vectors can be based on chimeric flaviviruses, including structural proteins (e.g., pre-membrane and envelope proteins) from a first flavivirus and non-structural proteins from a second, different flavivirus. The first and second flaviviruses can be, independently, selected from the group consisting of Japanese encephalitis, Dengue-1, Dengue-2, Dengue-3, Dengue-4, Yellow fever (e.g., YF17D), Murray Valley encephalitis, St. Louis encephalitis, West Nile, Kunjin, Rocio encephalitis, Ilheus, Tick-borne encephalitis, Central European encephalitis, Siberian encephalitis, Russian Spring-Summer encephalitis, Kyasanur Forest Disease, Omsk Hemorrhagic fever, Louping ill, Powassan, Negishi, Absettarov, Hansalova, Apoi, and Hypr viruses.

The heterologous sequences may include one or more influenza virus M2 or M2e sequences, or one or more immunogenic fragments or epitopes thereof. Further, the heterologous sequence(s) can optionally include a carboxy-terminal anchor-signal sequence, which may be from a flavivirus different from the flavivirus from which the envelope protein of the flavivirus vector is obtained. Further, the heterologous sequence(s) may optionally include one or more amino-terminal codons added to optimize cleavage (e.g., QP).

In the flavivirus vectors of the invention, the heterologous sequence(s) can include one or more immunogenic proteins, portions thereof, or immunologic epitopes thereof, of a viral, bacterial, fungal, or parasitic pathogen, or an oncogenic or allergenic protein.

Also included in the invention are chimeric flavivirus vectors that include structural proteins from a first flavivirus, non-structural proteins from a second, different flavivirus, and a heterologous sequence inserted at an intergenic site (i) between non-structural-2B (NS2B) and non-structural-3 (NS3) proteins of the chimeric flavivirus vector, or (ii) in the amino-terminal region of the polyprotein of the chimeric flavivirus vector. Such vectors may include pre-membrane and envelope proteins from the first flavivirus and capsid and non-structural proteins from the second, different flavivirus. The first and second flaviviruses can be, independently, selected from the group consisting of Japanese encephalitis, Dengue-1, Dengue-2, Dengue-3, Dengue-4, Yellow fever (e.g., YF17D), Murray Valley encephalitis, St. Louis encephalitis, West Nile, Kunjin, Rocio encephalitis, Ilheus, Tick-borne encephalitis, Central European encephalitis, Siberian encephalitis, Russian Spring-Summer encephalitis, Kyasanur Forest Disease, Omsk Hemorrhagic fever, Louping ill, Powassan, Negishi, Absettarov, Hansalova, Apoi, and Hypr viruses.

The heterologous sequence(s) can include one or more influenza virus M2 or M2e sequences, or one or more immunogenic fragments or epitopes thereof. The heterologous sequences may be flanked by protease cleavage sites, such as protease cleavage sites of YF17D. Further, the heterologous sequence(s) can be inserted in the amino terminal region of the flavivirus polyprotein, downstream from the polyprotein AUG, and/or downstream from the main cyclization signal of the vector. In addition, the vector polyprotein may be mutated and a new AUG may be present upstream from the heterologous sequence. The chimeric flavivirus vectors may also include an influenza virus mRNA translation enhancer. Examples of heterologous sequences that can be included in the vectors of the invention include any one or more of SEQ ID NOs:1-15, 17-41, and 43-79.

The invention also includes flavivirus vectors expressing one or more heterologous sequences inserted at an intergenic site in the amino terminal region of the flavivirus polyprotein, downstream from the main cyclization signal of the vector. In such vectors, the AUG of the vector polyprotein may be mutated and a new AUG may be present upstream from the heterologous sequence. Further, such vectors may include an influenza virus mRNA translation enhancer. In one example, such a flavivirus vector is based on yellow fever virus (e.g., YF17D) sequences. In a further example, the heterologous sequence(s) include an influenza virus M2 or M2e sequence, an immunogenic fragment or epitope thereof, or any other insert sequence as described herein or as is known in the art.

The invention further includes flavivirus replicons that include non-flavivirus sequences, such as influenza virus sequences (e.g., neuraminidase, hemagglutinin, M2, and/or M2e sequences, and/or one or more immunogenic fragments or epitopes thereof). In one example of the replicons of the invention, the non-flavivirus sequences replace the flavivirus pre-membrane and/or envelope sequences, which are provided for replication of the replicon in trans. In addition, the non-flavivirus sequences in the replicons of the invention can optionally include an element to produce the N-terminus of the non-flavivirus sequence.

Also included in the invention are pharmaceutical compositions that include one or more of the flavivirus vectors, chimeric flavivirus vectors, and/or replicons as described herein.

Further, the invention includes methods of delivering one or more heterologous sequences to a subject (e.g., a human subject), by administration of a pharmaceutical composition of the invention to the subject. In one example, the subject does not have, but is at risk of developing, infection by a pathogen (e.g., an influenza virus) from which the heterologous sequence is derived, or disease associated with a cancer antigen or allergen from which such sequence is derived. In another example, the subject is infected by a pathogen (e.g., an influenza virus) from which the heterologous sequence is derived, or has disease associated with the cancer antigen or allergen from which the heterologous sequence is derived.

The invention also includes methods of making flavivirus vectors, chimeric flavivirus vectors, and/or replicons as described herein, involving introducing a nucleic acid encoding the genome of the flavivirus vector, chimeric flavivirus vector, or replicon into a cell in which the flavivirus vector, chimeric flavivirus vector, or replicon replicates, and obtaining the flavivirus vector, chimeric flavivirus vector, or replicon from the cell or culture supernatant thereof.

The invention provides several advantages. For example, the live, attenuated viral vectors of the invention induce strong, long-lasting immune responses against specific antigens. The vectors of the invention can be used to confer immunity to infectious diseases, such as influenza, or to disease-related proteins such as cancer antigens and the like. As an example, the invention can be used to deliver influenza virus M2e (or a fragment thereof), which is the external portion of M2, a minor influenza A surface protein that is conserved among diverse influenza viruses and may serve as the basis for a vaccine that protects against all influenza A strains (Neirynck et al., Nat. Med. 5(10):1157-1163, 1999; Fiers et al., Virus Res. 103(1-2):173-176, 2004).

An additional advantage of the vectors of the invention is that, as described further below, they can be used to deliver relatively large antigens, as compared to many previously known viral vectors. Thus, as an example, in addition to M2e, the vectors of the invention can advantageously be used to administer larger portions of M2 or even full length M2.

The advantages of using live vectors, such as the flavivirus-based vectors of the invention, also include (i) expansion of the antigenic mass following vaccine inoculation; (ii) the lack of need for an adjuvant; (iii) the intense stimulation of innate and adaptive immune responses (YF17D, for example, is the most powerful known immunogen); (iv) the possibility of more favorable antigen presentation due to, e.g., the ability of chimeric flaviviruses (derived from YF17D) to infect antigen presenting cells, such as dendritic cells and macrophages; (v) the possibility to obtain a single-dose vaccine providing life-long immunity; (vi) the envelopes of chimeric flavivirus vaccine viruses are easily exchangeable, giving a choice of different recombinant vaccines, some of which are more appropriate than the others in different geographic areas or for sequential use; (vii) the possibility of modifying complete live flavivirus vectors into packaged, single-round-replication replicons, in order to eliminate the chance of adverse events or to minimize the effect of anti-vector immunity during sequential use; and (viii) the low cost of manufacture.

The possibility of easily exchanging the envelope proteins (the main antigenic determinants of immunity against flaviviruses) using chimeric flavivirus technology is a unique advantage. Several different vaccines can be constructed using the same YF 17D backbone (but different envelopes) that can be applied sequentially to the same individual, avoiding the problem of anti-vector immunity. On the other hand, different recombinant chimeric flavivirus insertion vaccines can be more appropriate for use in specific geographical regions in which different flaviviruses are endemic, as dual vaccines against an endemic flavivirus and another targeted pathogen. For example, a chimeric flavivirus including JE and influenza sequences may be more appropriate in Asia, where JE is endemic, to protect from both JE and influenza; YF 17D-influenza vaccine can be more appropriate for Africa and South America endemic for YF; a chimeric flavivirus including West Nile and influenza sequences, for the U.S. and parts of Europe and the Middle East in which WN virus is endemic; a chimeric flavivirus including dengue and influenza sequences, throughout the tropics where dengue viruses are present, etc. Yet, on the other hand, a chimeric flavivirus variant containing the envelope from a non-endemic flavivirus may be more desirable to avoid the risk of natural antivector immunity in a population that otherwise could limit the effectiveness of vaccination in a certain geographical area (e.g., a chimeric flavivirus including JE and influenza sequences may be preferable in the U.S. where JE is not present to more efficiently vaccinate people specifically against influenza).

Additional advantages provided by the invention relate to the fact that chimeric flavivirus vectors of the invention are sufficiently attenuated so as to be safe, and yet are able to induce protective immunity to the flaviviruses from which the proteins in the chimeras are derived and, in particular, the proteins or peptides inserted into the chimeras. Additional safety comes from the fact that some of the vectors used in the invention are chimeric, thus eliminating the possibility of reversion to wild type. An additional advantage of the vectors of the invention is that flaviviruses replicate in the cytoplasm of cells, so that the virus replication strategy does not involve integration of the viral genome into the host cell, providing an important safety measure. Further, a single vector of the invention can be used to deliver multiple epitopes from a single antigen, or epitopes derived from more than one antigen.

Other features and advantages of the invention will be apparent from the following detailed description, the drawings, and the claims.

### BRIEF DESCRIPTION OF THE DRAWINGS

FIG. 1 is an alignment of epitopes of influenza A virus M2e (A) (SEQ ID NOs:1-7) and HA₀ (B) (SEQ ID NOs:8-15) sequences.

FIG. 2 is an illustration of the flavivirus polyprotein.

FIG. 3 is an illustration showing that M2e expression at the E/NS1 junction in a chimeric flavivirus including JE pre-membrane and envelope sequences and yellow fever capsid and non-structural proteins should result in cell surface presentation of M2e, similar to NS1.

FIG. 4 is an illustration showing three variants used to express the full-length M2 protein at the N-terminus of the polyprotein precursor of a chimeric flavivirus including JE pre-membrane and envelope sequences and yellow fever capsid and non-structural proteins. Solid bars signify the 5′ sequences upstream and downstream the AUG start codon implicated in the cyclization of flavivirus RNA due to pairing with complementary nucleotides at the 3′ end (SEQ ID NO:16).

FIG. 5 is an illustration of a replicon based on a chimeric flavivirus including JE pre-membrane and envelope sequences and yellow fever capsid and non-structural proteins, expressing multiple influenza A virus immunogens as a multi-mechanism pandemic vaccine, e.g., expressing NA or HA in place of the prM-E genes, randomly inserted M2e epitope in, e.g., NS1, an immunodominant T-cell epitope in, e.g., NS3, and an additional immunogen(s) at inserted at one (or more) of the intergenic sites. The 2A autoprotease (from EMCV or FMDV) will cleave out NA from the rest of the polyprotein. Alternatively, and IRES element can be used instead of 2A autoprotease to re-initiate translation of NS proteins. A variety of elements (e.g., 2A autoprotease, ubiquitin, IRES, autonomous AUG for NA gene, or viral protease cleavage site) can be used to produce the N-terminus of NA at the site circled.

FIG. 6 is an illustration showing M2e-Eₜₘ, (transmembrane sequence containing the anchor-signal from E of YF 17D) cassettes inserted at the E/NS1 junction of a chimeric flavivirus including JE pre-membrane and envelope sequences and yellow fever capsid and non-structural proteins: without extra residues (upper panel; SEQ ID NOs:17 and 18), or with extra QP residues in front of M2e for optimal signalase cleavage (boxed; bottom panel; SEQ ID NOs: 19 and 20). The beginnings of M2e and the Eₜₘ sequences are indicated by arrow. Each fragment was produced using two long overlapping primers, which were annealed and filled-in with T4 DNA polymerase. The fragments were cloned directly (or after an additional PCR amplification step) by digestion with EheI (isoschizomer of NarI; the location of the sites in the fragments indicated) and ligation into the NarI site in the chimeric flavivirus genome (the 5.2 plasmid). Full-length DNA template for in vitro transcription was produced by standard two-fragment ligation. In vitro RNA transcripts were used to transfect Vero cells to generate virus.

FIG. 7 is an illustration showing expression of the M2e/YF 17D anchor-signal (A-S) cassette at the E/NS1 junction of a chimeric flavivirus including JE pre-membrane and envelope sequences and yellow fever capsid and non-structural proteins. Virus containing additional QP residues at the N-terminus of M2e was found viable as evidenced by the appearance of CPE after transfection, and staining of viral plaques with anti M2e MAb (right panel), but not the virus without the extra QP residues.

FIG. 8 shows genetic stability passages in Vero cells starting from the P2 stock of the QP-M2e virus at 37° C. Passage MOIs and apparent percentages of M2e positive virus are indicated.

FIG. 9 shows the stability of the insert in the QP-M2e virus during vertical generic stability passages from P2 or P5 (see in FIG. 8), at 37° C. (upper panels) or 34° C. (bottom panels), determined by RT-PCR amplification of M2e insert-containing region of the genome. At 37° C., the predominant (or the only band) is the insert-containing band of ˜600 nucleotides. At 34° C., the insert-containing band diminished, while shorter bands appeared with passages.

FIG. 10 shows examples of plaques of P7 viruses obtained starting from P2 (see in FIG. 8) at MOIs of 0.001 and 1 at 37° C., stained with JE and M2e specific antibodies. The numbers of plaques stained with the two antibodies are similar indicating high genetic stability. Following passages from P5 at 37° C., the staining pattern was similar. However, during passages at 34° C., the proportion of M2e-positive plaques significantly decreased as compared to the number of plaques stained with JE antibodies indicating that the virus is unstable at lower temperature.

FIG. 11 shows the immunogenicity of QP-M2e in Balb/c mice. (A) Survival of mock-immunized, QP-M2e virus-immunized, and HBc-M2e immunized (positive control) mice following IN challenge with 20 LD50 of influenza (PR8; a very high dose) is shown. (B) Dynamics of average post-challenge body weight indicative of morbidity is shown.

FIG. 12 shows two versions of an M2 insert flanked by the RRS viral protease cleavage sites, expressed at the NS2B/NS3 junction. The insert was cloned at an engineered AscI cloning site (the AscI sites flanking the insert are indicated). The RRS cleavage sites are underlined. The sequence upstream from the first RRS site represents the end of NS2B protein, and the sequence downstream from the second RRS site represents the beginning of NS3. The beginning (SLL . . . ) and the end ( . . . ELE) of the influenza M2 protein amino acid sequence are indicated by arrows (SEQ ID NOs:74-79).

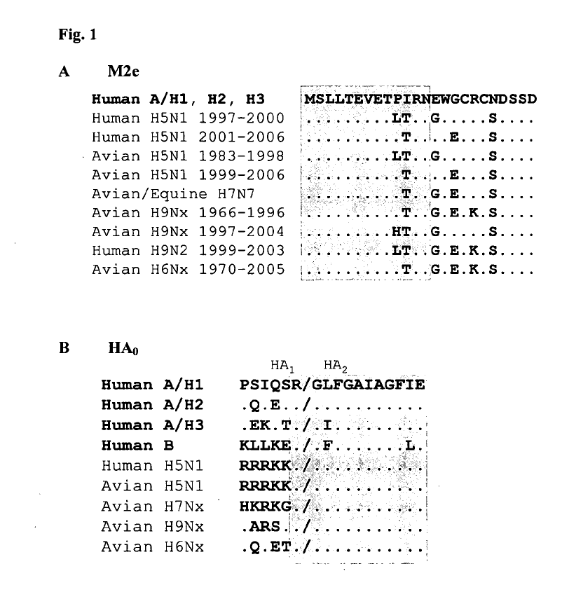

### DETAILED DESCRIPTION

This invention relates to the use of flavivirus, chimeric flavivirus, and replicon technology, as described further below, to create recombinant vaccines (including live vaccines) for the delivery of antigens such as influenza virus antigens and antigens of other pathogenic microorganisms. Live virus vaccines have significant advantages over subunit vaccines. In one example, chimeric flavivirus technology used in the invention is based on the YF17D vaccine virus, in which the premembrane and envelope (prM-E) protein genes are replaced with corresponding genes from a heterologous flavivirus. The safety and efficacy of vaccine candidates based on this technology has been demonstrated in multiple preclinical and clinical studies (Guirakhoo et al., Virology 257:363-372, 1999; Guirakhoo et al., J. Virol. 74:5477-5485, 2000; Guirakhoo et al., J. Virol. 75:7290-7304, 2001; Guirakhoo et al., Virology. 298:146-159, 2002; Guirakhoo et al., J. Virol. 78:4761-4775, 2004; Guirakhoo et al., J. Virol. 78:9998-10008, 2004; Monath et al., Vaccine 17:1869-1, 882, 1999; Monath et al., J. Virol. 74:1742-1751, 2000; Monath et al., Curr. Drug Targets Infect. Disord. 1:37-50, 2001; Monath et al., J. Virol. 76:1932-1943, 2002; Monath et al., Vaccine 20:1004-1018, 2002; Monath et al., J. Infect. Dis. 188:1213-1230, 2003; Monath et al., Vaccine 23:2956-2958, 2005; Monath et al., Biologicals 33:131-144, 2005; Monath et al., Proc. Natl. Acad. Sci. U.S.A. 103:6694-6699, 2006; Pugachev et al., Int. J. Parasitol. 33:567-582, 2003; Pugachev et al., Am. J. Trop. Med. Hyg. 71:639-645, 2004; Pugachev et al., in New Generation Vaccines, Third Edition, Levine et al. (Eds.), Marcel Dekker, New York, Basel, 3:559-571, 2004; Pugachev et al., Curr. Opin. Infect. Dis. 18:387-394, 2005).

Details of making chimeric viruses that can be used to make vectors of the invention are provided, for example, in U.S. Pat. Nos. 6,962,708 and 6,696,281; PCT international applications WO 98/37911 and WO 01/39802; Chambers et al., J. Virol. 73:3095-3101, 1999; and the references listed in the prior paragraph; the contents of each of which are incorporated by reference herein in its entirety.

As is discussed further below, any immunogenic proteins or epitopes from pathogenic viruses, bacteria, fungi, and/or parasites, as well as cancerogens and allergens, can be expressed in chimeric flaviviruses to create new recombinant human/veterinary vaccines against respective pathogens. The specific configuration of expression of a certain protein at a given expression site will depend on the properties of the protein expressed. Additional modifications can be carried out to increase genetic stability of recombinant vaccine virus and its immunogenicity, as is discussed further below.

The expression of the M2e immunogenic epitope of influenza A at the E/NS 1 junction of a chimeric flavivirus including pre-membrane and envelope proteins from a Japanese encephalitis virus, and capsid and non-structural proteins from a yellow fever virus (YF/JE chimera) is described below, and other expression sites can be used as well. As described further below, two new modifications were used for the E/NS1 site. First, the signalase cleavage site preceding M2e was optimized, to improve recombinant virus viability. Second, the anchor-signal sequences flanking M2e were chosen to be heterologous (i.e., taken from different viruses, JE and YF) to avoid the presence of direct nucleotide sequence repeats, and thus to reduce the chance of homologous recombination during virus replication, thereby increasing genetic stability of the recombinant virus (stability of the insert). The high stability of an M2e insert in the context of such a construct in vitro, which should be sufficient for manufacture, was demonstrated experimentally, as described further below.

In addition to the YF/JE chimera described above, additional chimeric flavivirus-based vectors can be used in the invention (e.g., chimeric flaviviruses including West Nile virus or Dengue virus (serotype 1, 2, 3, or 4) pre-membrane and envelope sequences, and yellow fever virus capsid and non-structural proteins; see, e.g., the references cited above). Other live vaccine viruses can be used as vectors instead of chimeric flavivirus-based vectors, including non-flavivirus live vaccines, such as alphavirus vaccine viruses (e.g., Venezuelan equine encephalomyelitis (VEE), Sindbis (SIN), and Semliki Forest (SFV) viruses), mononegaviruses (e.g., measles, rhabdoviruses, New Castle disease, Senadi, and parainfluenza viruses), rubella, retrovirus vectors, and attenuated strains of DNA viruses (e.g., vaccinia virus, smallpox vaccines, defective HSV vaccine viruses, adenoviruses, and adeno-associated viruses) (Shen and Post, in Fields Virology, Fifth Edition, Knipe et al. (Eds.), Wolters Kluwer and Lippincott Williams & Wilkins, Philadelphia, 539-564, 2007). In these vectors, particularly RNA virus vectors, foreign gene inserts can easily be designed and inserted intergenically, similar to as described below.

Further, other flaviviruses, such as non-chimeric flaviviruses, can be used as vectors according to the present invention. Examples of such viruses that can be used in the invention include live, attenuated vaccines, such the YF17D strain (and derivatives thereof), which was originally obtained by attenuation of the wild-type Asibi strain (Smithburn et al., “Yellow Fever Vaccination,” World Health Organization, p. 238, 1956; Freestone, in Plotkin et al. (eds.), Vaccines, 2^{nd} edition, W.B. Saunders, Philadelphia, U.S.A., 1995). An example of a YF17D strain from which viruses that can be used in the invention can be derived is YF17D-204 (YF-VAX®, Sanofi-Pasteur, Swiftwater, Pa., USA; Stamaril®, Sanofi-Pasteur, Marcy-L'Etoile, France; ARILVAX™, Chiron, Speke, Liverpool, UK; FLAVIMUN®, Berna Biotech, Bern, Switzerland; YF17D-204 France (X15067, X15062); YF17D-204, 234 US (Rice et al., Science 229:726-733, 1985)), while other examples of such strains that can be used are the closely related YF17DD strain (GenBank Accession No. U 17066), YF17D-213 (GenBank Accession No. U17067), and yellow fever virus 17DD strains described by Galler et al., Vaccines 16(9/10):1024-1028, 1998. In addition to these strains, any other yellow fever virus vaccine strains found to be acceptably attenuated in humans, such as human patients, can be used in the invention. Further, any other attenuated strains of flaviviruses belonging to the JE serocomplex (e.g., JE, WN, and Kunjin viruses), dengue serocomplex (DEN types 1-4 viruses), and TBE serocomplex (e.g., Langat and TBE viruses) can be used in the invention.

Any immunogenic proteins or appropriate portions thereof from pathogenic viruses, bacteria, fungi, and parasites, as well as cancerogens and allergens can be expressed intergenically in flaviviruses and chimeric flaviviruses (or their defective replicon variants) to create new recombinant human/veterinary vaccines against respective pathogens. The medical significance of one pathogen, influenza A virus, and how chimeric flavivirus vectors can be used to create influenza vaccines, as well as advantages of using chimeric flavivirus vectors, are described below. From this example, the applicability of the technology to developing vaccines against any other pathogens will become apparent, including improved vaccines for pathogens for which vaccines are available (e.g., tuberculosis and human papilloma virus), and new vaccines for pathogens for which no other vaccines are currently available (e.g., malaria, human immunodeficiency virus (HIV), herpes simplex virus (HSV), and hepatitis C virus (HCV)). While vaccines against seasonal influenza are available, the examples provided herein show that a universal influenza A vaccine can be developed using flavivirus and chimeric flavivirus vectors.

### Heterologous Peptides

The viral vectors of the invention can be used to deliver any peptide or protein of prophylactic or therapeutic value. For example, the vectors of the invention can be used in the induction of an immune response (prophylactic or therapeutic) to any protein-based antigen that is inserted into a virus vector (e.g., intergenically or into a virus protein, such as envelope, pre-membrane, capsid, and non-structural proteins of a flavivirus).

The vectors of the invention can each include a single epitope. Alternatively, multiple epitopes can be inserted into the vectors, either at a single site (e.g., as a polytope, in which the different epitopes can be separated by a flexible linker, such as a polyglycine stretch of amino acids), at different sites, or in any combination thereof. The different epitopes can be derived from a single species of pathogen, or can be derived from different species and/or different genuses.

Antigens that can be used in the invention can be derived from, for example, infectious agents such as viruses, bacteria, and parasites. A specific example of such an infectious agent is influenza viruses, including those that infect humans (e.g., A, B, and C strains), as well as avian influenza viruses. Examples of antigens from influenza viruses include those derived from hemagglutinin (HA; e.g., any one of H1-H16, or subunits thereof)(or HA subunits HA1 and HA2), neuraminidase (NA; e.g., any one of N1-N9), M2, M1, nucleoprotein (NP), and B proteins. For example, peptides including the hemagglutinin precursor protein cleavage site (HA0) (NIPSIQSRGLFGAIAGFIE (SEQ ID NO:21) for A/H1 strains, NVPEKQTRGIFGAIAGFIE (SEQ ID NO:22) for A/H3 strains, and PAKLLKERGFFGAIAGFLE (SEQ ID NO:23) for influenza B strains) or HA peptide SKAFSNCYPYDVPDYASL (SEQ ID NO:24), or its variant SKAFSNSYPYDVPDYASL (SEQ ID NO:25), or M2e (MSLLTEVETPIRNEWGSRSNDSSD (SEQ ID NO:26)) can be used; also see European Patent No. 0 996 717 B1, the contents of which are incorporated herein by reference), as well as peptide sequences listed in supplementary table 10 of Bui et al., Proc. Natl. Acad. Sci. U.S.A. 104:246-251, 2007, which can also be used (SEQ ID NOs:27-38). Other examples of peptides that are conserved in influenza can be used in the invention and include the NBe peptide conserved for influenza B (consensus sequence MNNATFNYTNVNPISHIRGS (SEQ ID NO:39)); the extracellular domain of BM2 protein of influenza B (consensus MLEPFQ (SEQ ID NO:40)); and the M2e peptide from the H5N1 avian flu (MSLLTEVETLTRNGWGCRCSDSSD (SEQ ID NO:41)).

Further examples of influenza peptides that can be used in the invention, as well as protein from which such peptides can be derived (e.g., by fragmentation) are described in US 2002/0165176, US 2003/0175290, US 2004/0055024, US 2004/0116664, US 2004/0219170, US 2004/0223976, US 2005/0042229, US 2005/0003349, US 2005/0009008, US 2005/0186621, U.S. Pat. No. 4,752,473, U.S. Pat. No. 5,374,717, U.S. Pat. No. 6,169,175, U.S. Pat. No. 6,720,409, U.S. Pat. No. 6,750,325, U.S. Pat. No. 6,872,395, WO 93/15763, WO 94/06468, WO 94/17826, WO 96/10631, WO 99/07839, WO 99/58658, WO 02/14478, WO 2003/102165, WO 2004/053091, WO 2005/055957, and Tables 1-4 (and references cited therein), the contents of which are incorporated by reference.

Protective epitopes from other human/veterinary pathogens, such as parasites (e.g., malaria), other pathogenic viruses (e.g., human papilloma virus (HPV), herpes simplex viruses (HSV), human immunodeficiency viruses (HIV), and hepatitis C viruses (HCV)), and bacteria (e.g., Mycobacterium tuberculosis, Clostridium difficile, and Helicobacter pylori) can also be included in the vectors of the invention. Examples of additional pathogens, as well as antigens and epitopes from these pathogens, which can be used in the invention are provided in WO 2004/053091, WO 03/102165, WO 02/14478, and US 2003/0185854, the contents of which are incorporated herein by reference.

Additional examples of pathogens from which antigens can be obtained are listed in Table 5, below, and specific examples of such antigens include those listed in Table 6. In addition, specific examples of epitopes that can be inserted into the vectors of the invention are provided in Table 7. As is noted in Table 7, epitopes that are used in the vectors of the invention can be B cell epitopes (i.e., neutralizing epitopes) or T cell epitopes (i.e., T helper and cytotoxic T cell-specific epitopes).

The vectors of the invention can be used to deliver antigens in addition to pathogen-derived antigens. For example, the vectors can be used to deliver tumor-associated antigens for use in immunotherapeutic methods against cancer. Numerous tumor-associated antigens are known in the art and can be administered according to the invention. Examples of cancers (and corresponding tumor associated antigens) are as follows: melanoma (NY-ESO-1 protein (specifically CTL epitope located at amino acid positions 157-165), CAMEL, MART 1, gp100, tyrosine-related proteins TRP1 and 2, and MUC1)); adenocarcinoma (ErbB2 protein); colorectal cancer (17-1A, 791Tgp72, and carcinoembryonic antigen); prostate cancer (PSA1 and PSA3). Heat shock protein (hsp110) can also be used as such an antigen.

In another example of the invention, exogenous proteins that encode an epitope(s) of an allergy-inducing antigen to which an immune response is desired can be used. In addition, the vectors of the invention can include ligands that are used to target the vectors to deliver peptides, such as antigens, to particular cells (e.g., cells that include receptors for the ligands) in subjects to whom the vectors administered.

The size of the peptide or protein that is inserted into the vectors of the invention can range in length from, for example, from 5-500 amino acids in length, for example, from 10-100, 20-55, 25-45, or 35-40 amino acids in length, as can be determined to be appropriate by those of skill in the art. Further, the peptides noted herein can include additional sequences or can be reduced in length, also as can be determined to be appropriate by those skilled in the art (e.g., by 1-10, 2-9, 3-8, 4-7, or 5-6 amino acids).

Whole foreign proteins, portions thereof, or individual immunogenic epitopes are expressed intergenically in the scope of this invention under the control of various functional elements to ensure viability of recombinant virus/replicon and optimal targeting of expressed antigen (intracellular, cell surface, extracellular delivery) necessary for the induction of robust immune response. These elements include appropriate signals, anchors, protease cleavage sites (both vector-virus specific and non-vector-virus specific), internal ribosome entry sites (IRES) elements, etc.

### Construction of the Vectors of the Invention

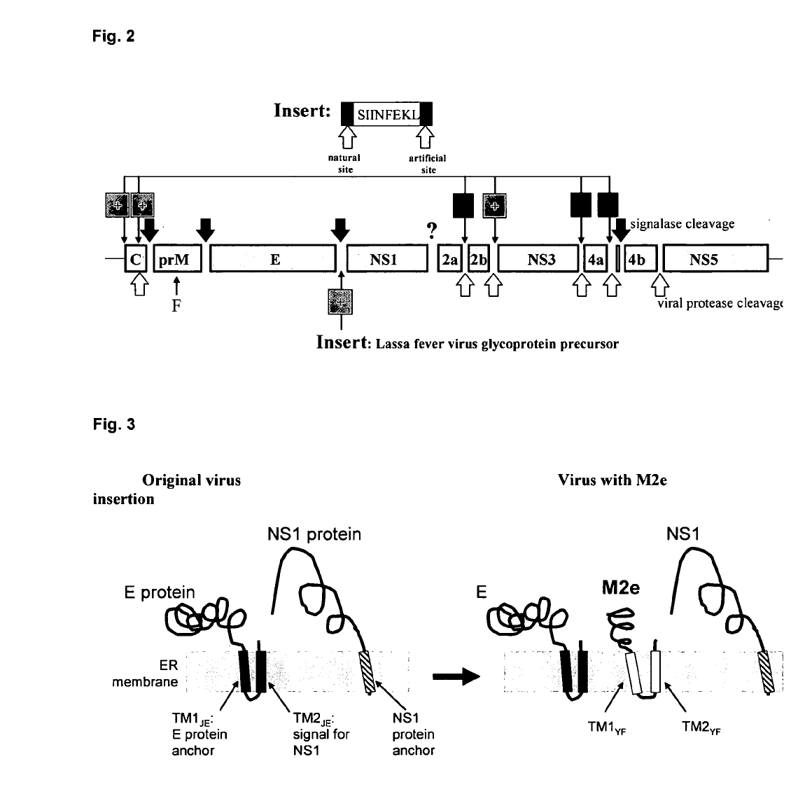

The organization of the flavivirus polyprotein precursor is shown in FIG. 2, and processing of the polyprotein yielding individual viral proteins is explained below. Also as is explained below, chimeric flaviviruses as used in the present invention can be designed as whole-virus vectors or as replicons.

Because the flavivirus prM, E, and NS1 glycoproteins are transported through the secretory pathway of the cell, expression of foreign proteins extracellularly (to be secreted) can be carried out by insertion at C/prM, prM/E, and E/NS1 junctions, and released from the polyprotein by signlase cleavages (similar to M, E, and NS1). Extracellular expression can be used when antibody (B-cell) responses to the expressed protein are desired. On the other hand, some proteins can be expressed cytoplasmically at other junctions and released by, e.g., viral protease cleavages, but still can be delivered to the cell surface or secreted. For example, the full-length M2 protein of influenza A virus synthesized in the cytoplasm will move towards the cytoplasmic membrane on its own; it will integrate into the cell membrane via the action of its hydrophobic domain located in the middle of the protein exposing the M2e etodomain on the cell surface. Other proteins can be forced to be secreted through the secretory pathway (endoplasmic reticulum/Golgi), when expressed at viral protease cleavage sites in the flavivirus polyprotein or the N-terminus of the polypotein, by using appropriate signal (membrane translocation) and anchor sequences at the N- and C-termini.

Further, viral structural proteins including glycoproteins of enveloped viruses (e.g., structural proteins of orthomyxoviruses, flaviviruses, rhabdoviruses, paramyxoviruses, filoviruses, and alphaviruses) when expressed alone or in a combination of two, three, etc., can be expected to form virus like particles (VLPs), which can be expected to be secreted. VLPs can be expected to be significantly more immunogenic as compared to individual immunogenic proteins. Thus, appropriate cassettes of genes of a heterologous virus can be expressed intergenically in flavivirus vectors (whole virus or replicon vectors) to produce VLPs. If T-cell (CTL) immunity is desired, foreign immunogens can be expressed cytoplasmically, e.g., at the N-terminus of the flavivirus polyprotein, or at the junctions cleaved by the viral protease (e.g., in the NS portion of the polyprotein), without the elements necessary for secretion. Once in the cytoplasm, the foreign proteins are processed and presented to the immune system via the MHC pathway. It should be noted that proteins destined for secretion also can be expected to be processed and presented via the MHC complex, resulting in T-cell responses (in addition to antibody responses).

In addition, gene shuffling technology can be used in the invention to achieve wide cross-protective immunity against multiple strains/genotypes/serotypes of a target pathogen (see, e.g., Locher et al., DNA Cell Biol. 24(4):256-263, 2005). In particular, immunogenic protein(s) of interest from different strains of one pathogen (e.g., influenza, HIV, HCV, dengue, and rhinovirus) can be reshuffled, and then a reshuffled gene or gene cassette can be expressed in a flavivirus vector to obtain a widely (or universally) protective vaccine conferring immunity against multiple (or all) strains/types of a target pathogen.

### Insertion of Influenza Epitopes at Gene Junctions in the Polyprotein Open Reading Frame of Flavivirus Vaccine Viruses

The M2e peptide or the full-length M2 protein can be expressed, properly positioned at the protein junctions in the flavivirus polyprotein precursor, such that: i) the expression products are delivered to the cell surface, and ii) vector virus replication is not compromised. M2 is a type 3 integral membrane protein with no cleavable N-terminal signal. In addition to the 23-amino acid N-terminal ectodomain (M2e), the protein contains a 19-amino acid transmembrane domain, and a 54-amino acid cytoplasmic tail. The protein forms tetrameric ion channels on the surfaces of virus-infected cells and viral particles (Lamb et al., in Fields. Virology, Fourth Edition, Knipe (Ed.), Lippincott Williams and Wilkins, Philadelphia, 1043-1126, 2001). Thus, the complete M2 protein expressed cytoplasmically by, e.g., a chimeric flavivirus (e.g., a YF/JE chimera, as described herein), should be naturally directed to the cell surface. Its correct N- and C-termini can be produced in the cytoplasm by the vector virus NS2B/NS3 protease. The ion channel activity can be turned off, if desired, by growing recombinant virus in the presence of amantadine or by specific mutations/deletions in the 19-amino acid hydrophobic α-helix (McCown et al., J. Virol. 79:3595-3605, 2005). The cytoplasmic tail of M2, as well as some portions of the transmembrane domain, appear to be unnecessary for cell surface presentation of the M2e ectodomain (McCown et al., J. Virol. 79:3595-3605, 2005; Watanabe et al., J. Virol. 75:5656-5662, 2001). Moreover, M2e attached to a heterologous anchor (e.g., the Sendai virus F protein, which is a classical type 1 membrane glycoprotein) was found to be delivered to the cell surface (Park et al., J. Virol. 72:2449-2455, 1998). Thus, M2e expressed between flavivirus glycoproteins (type 1 membrane proteins) can be targeted to the cell surface. The HA₀ epitope can be similarly targeted to the cell surface.

Description of three examples of flavivirus vectors of the invention follow.

1) Expression of M2e at the E/NS1 junction. Expression of M2e in a format similar to flavivirus NS1 (or E) protein is expected to result in abundant presentation of the peptide on the cell surface. This can be carried out by inserting an M2e/E protein anchor/signal cassette at the E-NS1 junction of a chimeric flavivirus (YF/JE). The principle is illustrated in FIG. 3. The JE-specific E, YF 17D-specific NS1, as well as anchored M2e in between, should be translocated into the lumen of the ER, and the N-termini of each of the three individual proteins should be released by signalase cleavages. To exclude the possibility of homologous recombination that can reduce insert stability during virus replication, the additional signal/anchor sequence (transmembrane domains 1 and 2, TM1 and TM2) from the C-terminus of the E protein of YF 17D can be used. This fragment differs significantly at the nucleotide sequence level from its analog at the end of JE-specific E protein gene in YF/JE vector.

To ensure efficient signalase cleavage at the E_{JE}/M2e junction, the cleavage site can be optimized by using additional residues at the N-terminus of M2e (e.g., using the SignalP 3.0 program available on-line). The complete M2 protein can also be expressed at this location. Cell surface delivery of M2e can also be carried out by using other anchor-signal sequences (taken from other flaviviruses, e.g., wild type dengue, or non-flavivirus sequences); alternatively M2e can be attached to the N-terminus of an appropriate glycoprotein carrier that will facilitate cell surface delivery/secretion of M2e in its native linear conformation. Such a carrier can be immunologically inert, it can be chosen to induce a desired immune response (against influenza or against a heterologous pathogen), or it can have an immunostimulatory function (e.g., if it is a cytokine or a TLR agonist, etc.). Additional information concerning a chimeric flavivirus including such an insertion at the E/NS1 junction is provided below, in the Experimental Results section.

2) Expression of full-length M2 at the NS2B/NS3 junction. The M2 protein flanked by YF 17D protease cleavage sites can be inserted at the NS2B/NS3 junction of a chimeric flavivirus including JE pre-membrane and envelope sequences and yellow fever capsid and non-structural sequences (see FIG. 2). The protein should be released from the polyprotein in the cytoplasm and is expected to be transported to the cell surface in its native tetrameric form. If desired, the ion channel activity of M2 can be turned off to ensure efficient recombinant virus replication and/or genetic stability. Additional information concerning a chimeric flavivirus including such an insertion at the N2SB/NS3 junction is provided below, in the Experimental Results section.

3) Expression of full-length M2 at the N-terminus of the viral polyprotein. The full-length M2 can also be expressed at the N-terminus of the chimeric flavivirus (e.g., a YF/JE chimera) polyprotein for cell surface presentation (see FIG. 2). An important requirement for viability is that M2 is cleaved out from the rest of the viral polyprotein, such that the N-terminus of the capsid protein C remains in the cytoplasm. This can be achieved by adding a viral NS2B/NS3 protease cleavage site at the M2/C protein junction (McAllister et al., J. Virol. 74:9197-9205, 2000). Because of the uncertainty of the effect of M2 gene insertion at this location on the cyclization of flavivirus RNA, and in order to explore the effect of a translational enhancer found in influenza virus mRNAs (Kash et al., J. Virol. 76:10417-10426, 2002), three different constructs are proposed.

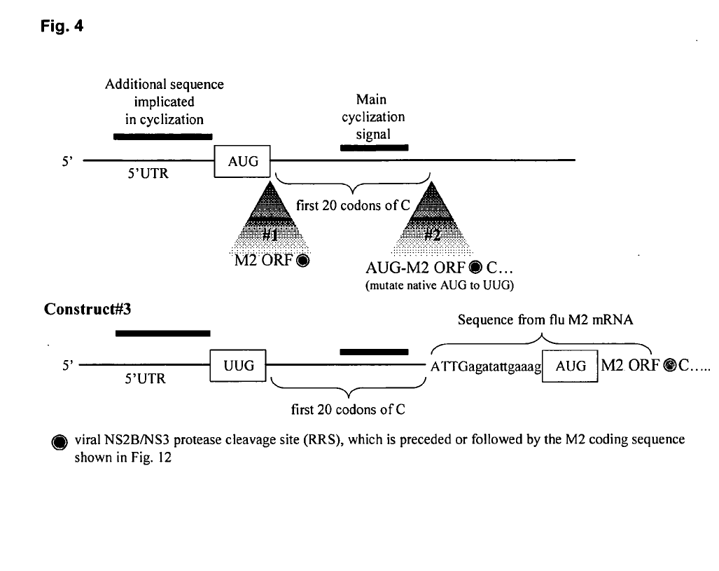

First, we insert the M2 gene/viral protease site downstream from the viral start codon (Construct #1 in FIG. 4). The M2 insert (96 amino acids, 288 nucleotides) results in separation of the sequences upstream and downstream from the AUG start codon (FIG. 4) that have been implicated in cyclization of flavivirus RNA (Alvarez et al., J. Virol. 79(11):6631-6643, 2005). The cyclization occurs due to interactions of complementary nucleotides at the 5′ and 3′ ends of the genome and is critical for viral RNA synthesis and possibly translation (Khromykh et al., J. Virol. 75:6719-6728, 2001; Edgil et al., J. Virol. 80:2976-2986, 2006; Khromykh et al., J. Virol. 75:6719-6728, 2001; Nomaguchi et al., J. Biol. Chem. 279:12141-12151, 2004; Shurtleff et al., Virology 281:75-87, 2001).

In Construct #2, to avoid the separation, the native AUG start codon is ablated (e.g., changed to UUG) and the M2 insert, starting with its own AUG codon, is placed downstream from the main cyclization signal located within the first 20 codons of the C gene (not translated in Construct #2). A viral protease cleavage site is placed downstream from the M2 gene, followed by the viral open reading frame (ORF). In the ORF, the main cyclization signal can be ablated, to avoid its repetition, using degenerate codons. To increase translation, the AGGT (SEQ ID NO:42) translational enhancer found in influenza virus mRNAs (Kash et al., J. Virol. 76:10417-10426, 2002) can optionally be inserted into Construct #2, between the main cyclization signal and the M2 gene, resulting in Construct #3 (FIG. 4). The enhancer increases translation of influenza virus mRNAs, possibly contributing to host cell translational shut off observed following influenza virus infection. A variety of molecular techniques are known in the art for use in making these constructs (e.g., overlap PCR and site-directed mutagenesis, with and without subcloning). However, to simplify the plasmid construction process, the 5′ fragments containing the proposed modifications can be synthesized commercially (e.g., custom gene synthesis by DNA 2.0, Inc.) and introduced into a chimeric flavivirus infectious clone using available convenient restriction sites.

Chimeric Flavivirus-Based Replicons as a Vaccines Inducing Protection from Flu Via Multiple Immunological Mechanisms (or Against a Different Pathogen, or Several Different Pathogens)

Single-round replicon technology can also be used in the present invention. Such technology is well established for flaviviruses, and the immunogenic potential of recombinant replicons has been demonstrated (Jones et al., Virology 331:247-259, 2005; Molenkamp et al., J. Virol. 77:1644-1648, 2003; Westaway et al., Adv. Virus. Res. 59:99-140, 2003; Herd et al., Virology 319:237-248, 2004; Harvey et al., J. Virol. 77:7796-7803, 2003; Anraku et al., J. Virol. 76:3791-3799, 2002; Varnayski et al., J. Virol. 74:4394-4403, 2000). In a replicon, the prM and E protein genes are deleted, as well as a C-terminal portion of C. Therefore, the replicon can replicate inside cells but cannot generate virus progeny (hence single-round replication). It can be packaged into viral particles when the prM-E (and if necessary, C) genes are provided in trans. Still, when cells are infected by such packaged replicon (e.g., following vaccination), a single round of replication follows, without further spread to surrounding cell/tissues. Using packaged replicon particles (expressing foreign proteins/epitopes) as vaccines is advantageous, because the particle itself provides strong immune stimulation, as was shown for YF 17D (Querec et al., JEM 203:413-424, 2006; Palmer et al., J. Gen. Virol. 88:148-156, 2007). Alternatively, immunization can be achieved by inoculation of the replicon in the form of naked DNA or RNA.

Although chimeric flavivirus-based vaccines are safe and effective, avoiding systemic replication of a recombinant vaccine virus may be desirable to increase safety. The latter can be achieved by using the replicon approach. The use of replicons should minimize problems with antivector immunity, e.g., in persons naturally immune to the whole virus or immune through vaccination (due to the presence of neutralizing antibodies), as well as in order to use the same vector for production of different vaccines that can be given sequentially to the same individual. Replicons also offer the opportunity to express a larger number of immunogenic moieties. Chimeric flavivirus (e.g., YF/JE chimera)-based replicons can be constructed expressing an influenza virus immunogenic polypeptide (e.g., NA or HA) in place of the C-prM-E structural protein genes (while keeping the cyclization signal intact), or only the prM-E envelope protein genes. Additional antigenic determinants can be incorporated into the replicon NS proteins, or at an appropriate gene junction (FIG. 5). Chimeric flavivirus-flu replicons can be packaged into viral particles by supplying the prM-E or C-prM-E proteins in trans. This can be done using, e.g., alphavirus replicons (such as VEE, Sindbis, or SFV replicons) or via stable packaging cell lines (e.g., as in Mason et al., Virology 351:432-443, 2006).

In addition to its potential as a stand-alone vaccine, a chimeric flavivirus-flu (or other) replicon (or recombinant virus) can be used in combination with a non-replicating vaccine component such as a HBc-M2e subunit vaccine (Fiers et al., Virus Res. 103:173-176, 2004; Neirynck et al., Nat. Med. 5:1157-1163, 1999). We have found that co-inoculation of HBc-M2e together with whole YF 17D resulted in immune responses and, thus, YF 17D or chimeric flavivirus-based replicons can be expected to function as natural adjuvants. The latter feature is highly desirable, because IgG2a antibodies are significantly more active than IgG1 in ADCC, the principal mechanism of the anti-M2 immunity. In addition to improved antibody response to HBc-M2e, the replicon can be used to deliver other antigenic determinants.

Further, in whole-virus chimeric flavivirus-based recombinants, as well as in replicon recombinants, the chimeric flavivirus genome can be rearranged, thus providing additional options for recombinant vaccine design. For example, one, some, or all of the C-prM-E structural proteins can be transferred to the 3′ end of the genome and expressed after NS5, under the control of an IRES element (e.g., as recently described for TBE virus; Orlinger et al., J. Virol. 80:12197-12208, 2006), or by using separation from NS5 (if expressed in-frame) via a cleavage by viral or an appropriate non-viral protease at an engineered cleavage site. Such rearrangement can confer some advantages, e.g., an increase in the degree of attenuation.

Following construction of recombinant viruses/replicons of the invention, expression of influenza immunogens can be ascertained by a variety of available methods, such as immunoblots, immunofocus assay, ELISA, etc. The recombinants/replicons can be tested for safety, immunogenicity in vivo, and the ability to provide protection from flu challenge (many animal models are available such as mice, hamsters, non-human primates), and genetic stability in vitro and in vivo.

### Production and Administration

The viral vectors described above can be made using standard methods in the art. For example, an RNA molecule corresponding to the genome of a virus can be introduced into primary cells, chicken embryos, or diploid cell lines, from which (or the supernatants of which) progeny virus can then be purified. Other methods that can be used to produce the viruses employ heteroploid cells, such as Vero cells (Yasumura et al., Nihon Rinsho 21:1201-1215, 1963). In an example of such methods, a nucleic acid molecule (e.g., an RNA molecule) corresponding to the genome of a virus is introduced into the heteroploid cells, virus is harvested from the medium in which the cells have been cultured, harvested virus is treated with a nuclease (e.g., an endonuclease that degrades both DNA and RNA, such as Benzonase™; U.S. Pat. No. 5,173,418), the nuclease-treated virus is concentrated (e.g., by use of ultrafiltration using a filter having a molecular weight cut-off of, e.g., 500 kDa), and the concentrated virus is formulated for the purposes of vaccination. Details of this method are provided in WO 03/060088 A2, which is incorporated herein by reference. Further, methods for producing chimeric viruses are described in the documents cited above in reference to the construction of chimeric virus constructs.

The vectors and replicons of the invention are administered in amounts and by using methods that can readily be determined by persons of ordinary skill in this art. In the case of chimeric flaviviruses and yellow fever virus-based vectors, the vectors can be administered and formulated, for example, in the same manner as the yellow fever 17D vaccine, e.g., as a clarified suspension of infected chicken embryo tissue, or a fluid harvested from cell cultures infected with the chimeric yellow fever virus. The vectors of the invention can thus be formulated as sterile aqueous solutions containing between 100 and 1,000,000 infectious units (e.g., plaque-forming units or tissue culture infectious doses) in a dose volume of 0.1 to 1.0 ml, to be administered by, for example, intramuscular, subcutaneous, or intradermal routes (see, e.g., WO 2004/0120964 for details concerning intradermal vaccination approaches). In addition, because flaviviruses may be capable of infecting the human host via the mucosal routes, such as the oral route (Gresikova et al., “Tick-borne Encephalitis,” In The Arboviruses, Ecology and Epidemiology, Monath (ed.), CRC Press, Boca Raton, Fla., 1988, Volume IV, 177-203), the vectors can be administered by a mucosal route.

When used in immunization methods, the vectors and replicons can be administered as a primary prophylactic agent in adults or children at risk of infection by a particular pathogen. The vectors and replicons can also be used as secondary agents for treating infected patients by stimulating an immune response against the pathogen from which the peptide antigen is derived.

For vaccine applications, optionally, adjuvants that are known to those skilled in the art can be used. Adjuvants that can be used to enhance the immunogenicity of the vectors include, for example, liposomal formulations, synthetic adjuvants, such as (e.g., QS21), muramyl dipeptide, monophosphoryl lipid A, or polyphosphazine. Although these adjuvants are typically used to enhance immune responses to inactivated vaccines, they can also be used with live vaccines. In the case of a vector delivered via a mucosal route, for example, orally, mucosal adjuvants such as the heat-labile toxin of E. coli (LT) or mutant derivations of LT can be used as adjuvants. In addition, genes encoding cytokines that have adjuvant activities can be inserted into the vectors. Thus, genes encoding cytokines, such as GM-CSF, IL-2, IL-12, IL-13, or IL-5, can be inserted together with foreign antigen genes to produce a vaccine that results in enhanced immune responses, or to modulate immunity directed more specifically towards cellular, humoral, or mucosal responses. In addition to vaccine applications, as those skilled in the art can readily understand, the vectors of the invention can be used in gene therapy methods to introduce therapeutic gene products into a patient's cells and in cancer therapy.

### Experimental Results

### Expression of the M2e Epitope at the E/NS1 Junction of a Chimeric Flaivirus (YF/JE)

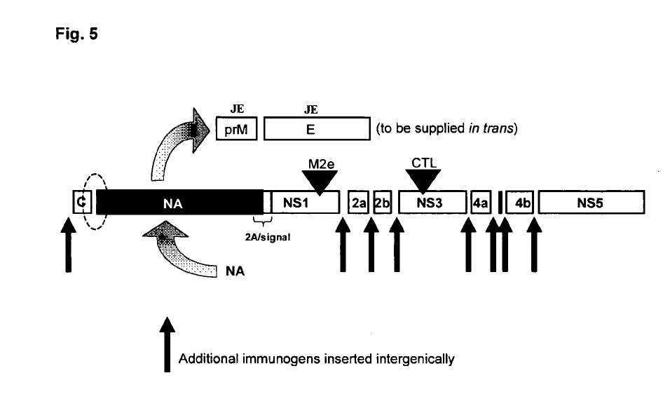

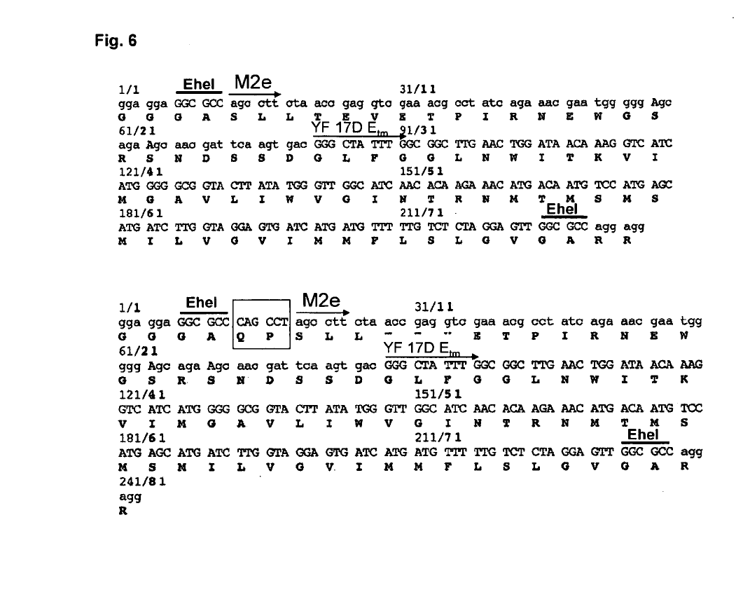

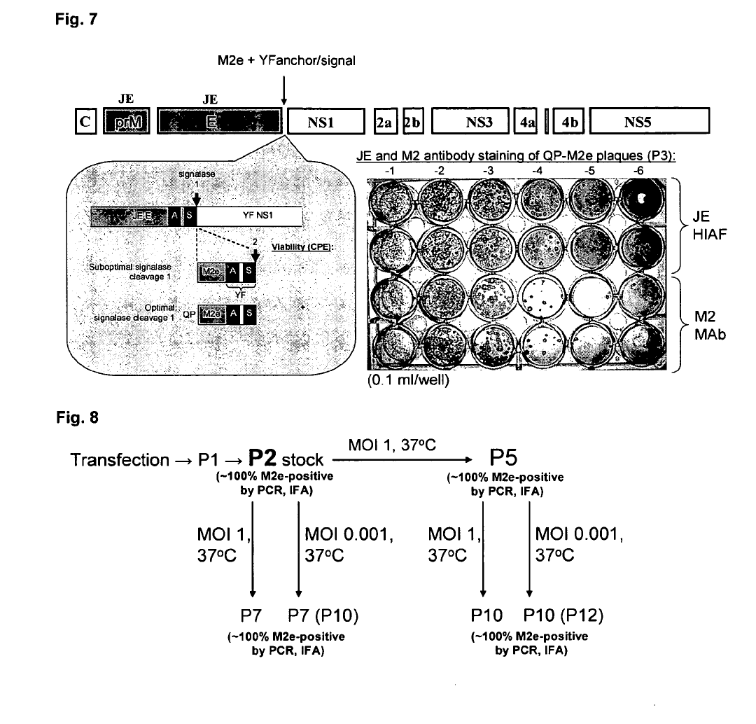

A cassette encoding the M2e peptide fused with the transmembrane domain from the C-terminus of the E protein of YF 17D (FIG. 6; anchor-signal, A-S in FIG. 7) was inserted at the E/NS1 junction of a chimeric flavivirus (YF/JE) (FIG. 7). In this construct, the N-terminus of M2e should be generated by a signalase cleavage, similar to the N-terminus of NS1 in the parental virus. The transmembrane domain is necessary for proper anchoring of the M2e peptide and translocation of the downstream NS1 into the lumen of endoplasmic reticulum. The M2e peptide is expected to be delivered to the cell surface and possibly secreted. The rationale for using the YF 17D specific anchor-signal, rather than JE-specific anchor-signal (present in the vector virus), is to reduce the chance of homologous recombination during replication of the resulting recombinant virus, increasing the stability of M2e insert.

A second variant of this expression construct was engineered to contain two extra residues, Q and P, at the N-terminus of M2e. This addition was computer-predicted to result in a more efficient signalase cleavage at the E_{JE}/M2e junction. (Specifically, the probability of cleavage at E/M2e without QP was predicted by the SignalP 3.0 program (www.cbs.dtu.dk/services/SignalP) to be 0.454, while the predicted probability of cleavage at the native E/NS1 site in a chimeric flavivirus including JE pre-membrane and envelope sequences and yellow fever capsid and non-structural sequences is 0.663. The addition of QP increased the probability of cleavage at the E/M2e junction to 0.807).

Following transfection of Vero cells with in vitro synthesized RNA transcripts, the QP-M2e variant, but not the variant without QP, was found to be viable. It produced CPE, and transfected cells were efficiently stained with the M2e MAb, as were viral plaques at P2 passage (FIG. 7) (P2 was obtained by amplification in Vero cells of the P1 viral sample harvested after transfection). The same number of plaques were stained with anti-JE antibodies (mouse hyperimmune ascitic fluid, HIAF; FIG. 7), indicating that the P2 virus stock was homogeneous in terms of the presence of the M2e insert. Thus, this experiment demonstrated feasibility of using the E/NS1 junction for the expression of an antigenic determinant in a chimeric flavivirus including JE pre-membrane and envelope sequences and yellow fever capsid and non-structural sequences.

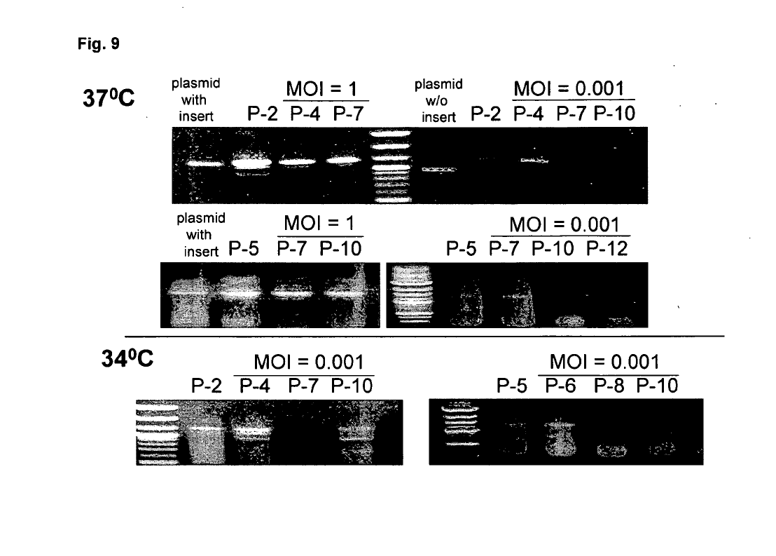

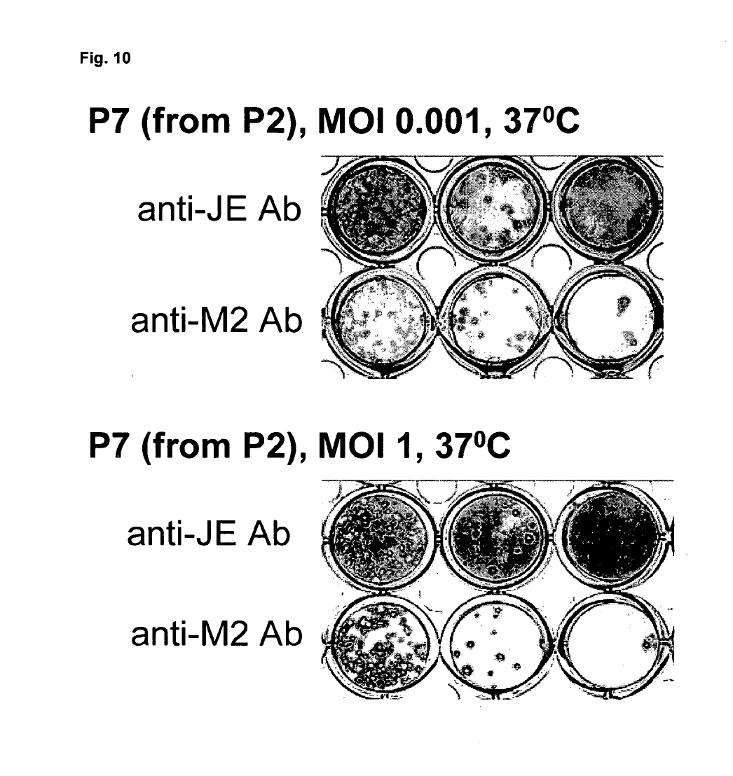

We investigated in vitro genetic stability of the QP-M2e variant by propagating the virus in Vero cells in a series of passages as shown in FIG. 8. The P2 stock was first passaged to P5 (horizontal passages in FIG. 8; estimated MOI˜1 (undiluted virus)). Both viruses appeared to be 100% homogeneous in terms of the presence of the insert and M2e expression, as determined by RT-PCR (see P2 and P5 bands in FIG. 9) and immunofocus assay (IFA) using staining with anti-JE or anti-M2e antibodies, respectively. The P2 and P5 samples were further passaged five times (and in some cases 7 times) at MOI 1 or 0.001 at 37° C. (vertical passages in FIG. 8). The final passage samples were also found to be predominantly M2e positive by RT-PCR (FIG. 9, upper panels) and IFA (examples of plaques of P7 viruses produced from P2 starting virus at MOIs 0.001 and 1 at 37° C. shown in FIG. 10). All samples had high titers of 6-7 log₁₀ pfu/ml (as determined by staining with both M2e-MAb and anti-JE HIAF), demonstrating that the QP-M2e virus replicate efficiently in vitro. (The vertical passages were also done at 34° C. The proportion of insert-containing virus progressively decreased, as demonstrated by the appearance of shorter RT-PCR bands (exemplified for MOI 0.001 in FIG. 9, bottom panels), and accumulation of M2e-negative plaques in IFA which at the final passages constituted up to ˜50-70% of all plaques. Thus, cell growth conditions, such as lower temperature, can affect genetic stability).

The QP-M2e virus has a sufficiently high genetic (insert) stability for manufacture. For example, if the P2 stock virus were a pre-Master Seed (PMS) virus, only three more passages would be necessary to manufacture a final vaccine lot (Master Seed, Production Seed, and vaccine lot passages. This genetic stability experiment demonstrates that the virus (when grown in optimal conditions) is stable for at least 5 low or high MOI passages, or 5 high-MOI+5 low-MOI passages, or 10 high MOI passages.

Genetic stability of the QP-M2e recombinant may be further increased, if desired, by plaque purification. A new cloned (plaque-purified) viral stock can be produced and similarly tested for the stability of the insert and M2e expression in multiple passages in vitro. In addition, if the size of the insert is found to play a role in stability, because of the increase of the overall size of viral genomic RNA (which may result in less efficient packaging and thus a reduced genetic stability of the recombinant due to selective pressure), genetic stability can be increased by introducing a benign deletion elsewhere. For example, in our recent studies, we have demonstrated that a chimeric flavivirus including West Nile pre-membrane and envelope protein sequences and yellow fever virus capsid and non-structural sequences virus can tolerate a 147-nucleotide deletion in the beginning of the 3′UTR without a marked effect on replication in vitro and in vivo and immunogenicity (WO 06/116182). Such a deletion may be used in the present invention as well. In addition, a more stable variant, e.g., containing a silent nucleotide change(s) in the insert and/or in surrounding viral sequences may be isolated from the population at a late genetic stability passage. The change(s) may stabilize the insert by further decreasing the chances of homologous recombination.

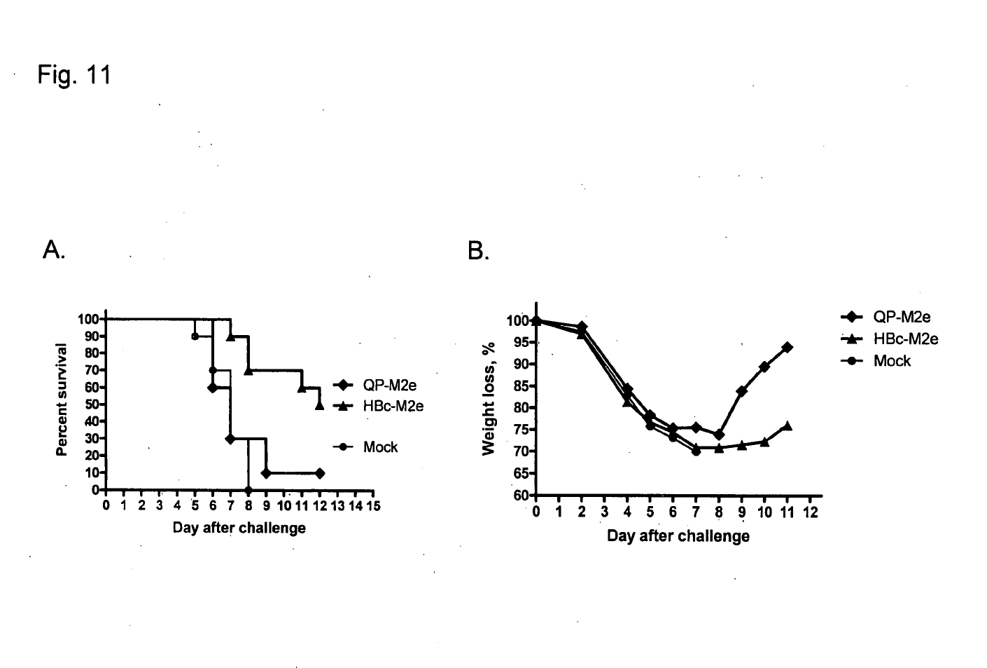

In FIGS. 7 and 10, plaques of the QP-M2e virus were visualized by anti-M2e monoclonal antibody staining after methanol fixation of the cell monolayer. Methanol treatment permeabilizes cell membranes, allowing the Mab to interact with intracellular protein, and to detect antigen inside the cells, in this case in viral plaques. In one experiment, when cells were not pretreated with methanol prior to incubation with M2e Mab, no immunostaining of plaques was observed. Therefore, it appears that the expressed M2e peptide did not reach the cell surface. Cell-surface presentation can be improved using specific signals efficiently targeting peptides/proteins to the cell surface. In agreement with the above observation, when Balb/c mice were immunized subcutaneously (SC) with 5 log₁₀ pfu of QP-M2e virus, and boosted on day 40, antibody responses to M2e determined in pooled mouse sera by ELISA on day 54 were low. Nevertheless, when immunized mice were challenged on day 55 intranasally with 20 LD50 of mouse-adapted influenza virus (strain A/PR8/64), there were signs of protection. All ten mock-immunized animals (negative control) lost weight, became sick, and died very quickly, with an average survival time (AST) of 6.9 days. Animals immunized with QP-M2e virus lost weight more slowly and one animal eventually recovered (FIGS. 11A and 11B). In the positive control, where mice were immunized twice SC with HBc-M2e (10 μg/dose with Alum), animals also lost weight after challenge, and 5 out of 10 animals died (FIG. 11). Importantly, weight loss, which is an indicator of morbidity, was more pronounced in the HBc-M2e control group, as compared to the QP-M2e group. It is possible that low-level M2e specific antibodies were present in QP-M2e immunized animals conferring a degree of protection. In addition, the M2e peptide also contains a CTL epitope (Fiers et al., Virus Research 103:173-176, 2004), which could also mediate protection. It should be noted that the challenge dose of influenza virus in this experiment (20 LD50) may have been too high. Had we used a lower challenge dose (e.g., 4-10 LD50, which is common in protection experiments in which M2e is used as immunogen), better protection could have been observed.

In addition, we have recently established that an intraperitoneal (IP) prime/IP boost immunization regimen (dose 10⁷ log₁₀ pfu) is more efficient for immunizing mice with chimeric flavivirus-M2e recombinant viruses, and could provide a better demonstration of immunogenicity of QP-M2e virus. Further, mice are poor responders to chimeric flavivirus immunization due to relatively inefficient virus replication in vivo in this model, and thus a much higher immunogenicity/efficacy could be expected in the more sensitive primate models (non-human primates and humans).

### Expression of the M2 Gene at the NS2B/NS3 Junction of a Chimeric Flavivirus (YF/JE)

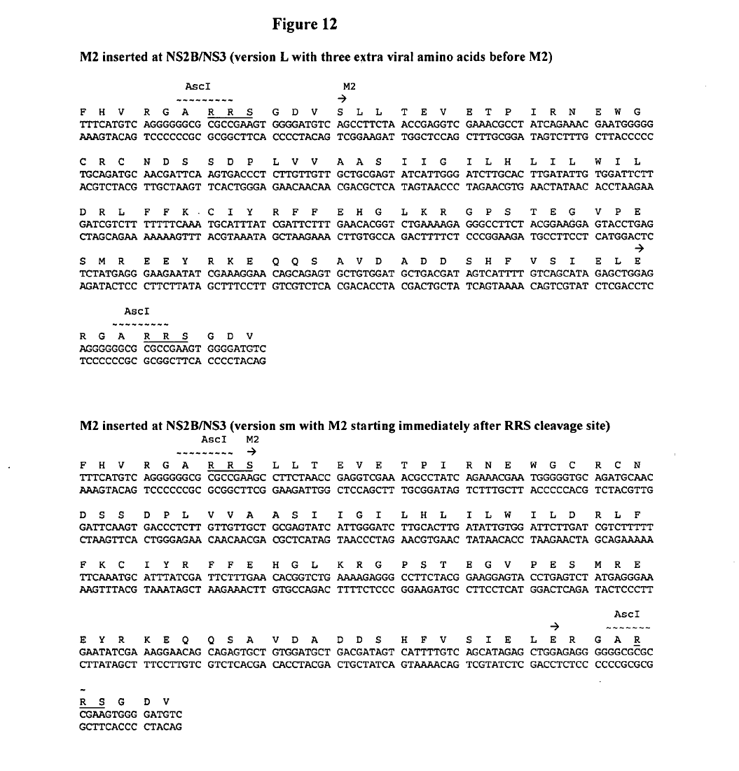

In additional studies, a unique AscI restriction site was introduced at the NS2B/NS3 gene junction in a chimeric flavivirus (YF/JE) by silent mutagenesis. Full-length M2 gene of influenza A virus flanked by viral (YF 17D) protease cleavage sites (RRS) was inserted at the AscI site Two engineered versions of the insertion are shown in FIG. 12. The difference between the first version (L) and the second version (sm) was the presence or absence, respectively, of three viral residues (GDV) upstream from the M2 coding sequence. After the first attempt to generate virus by transfecting Vero cells with in vitro RNA transcripts, all plaques in harvested P1 progeny virus were found to be M2-negative. It is possible that M2 changes the cellular environment due to its ion-channel activity, which creates a selective pressure on M2-containing recombinant, resulting in quick accumulation of M2-negative vector virus. If true, this can be overcome by growing the virus in the presence of amantadine (inhibitor of M2 ion-channel activity), or by introducing specific mutations in the transmembrane region of M2 to turn off the ion-channel activity.

TABLE 1
Influenza A virus CTL Epitopes of the Nucleoprotein

| Amino Acid Positions (ref.) | Host | MHC restriction |
|---|---|---|
| 44-52 (ref. 14) | Human | HLA-A1 |
| 50-63 (ref. 3) | Mouse (CBA) | H-2Kk |
| 91-99 (ref. 13) | Human | HLA-Aw68 |
| 147-158 (ref. 5) | Mouse (Balb/c) | H-2Kd |
| 265-273 (ref. 14) | Human | HLA-A3 |
| 335-349 (ref. 1) | Human | HLA-B37 |
| 335-349 (ref. 2) | Mouse | HLA-B37 |
| 365-380 (ref. 2) | Mouse | H-2Db |
| 366-374 (ref. 9) | Mouse (C57B1/6) | H-2Db |
| 380-388 (ref. 16) | Human | HLA-B8 |
| 383-391 (ref. 16) | Human | HLA-B27 |

TABLE 2
Influenza A virus T helper Epitopes of the Nucleoprotein

| Amino Acid Positions (ref.) | Host | MHC restriction |
|---|---|---|
| 55-69 (ref. 8) | Mouse (Balb/c) | H-2Kd |
| 182-205 (ref. 11) | Human | |
| 187-200 (ref. 8) | Mouse (CBA) | H-2Kk |
| | Mouse (Balb/c) | H-2Kd |
| 216-229 (ref. 8) | Mouse (Balb/c) | H-2Kd |
| 206-229 (ref. 11) | Human | HLA-DR1, HLA-DR2 en |
| | | HLA-DRw13 |
| 260-283 (ref. 8) | Mouse (CBA) | H-2Kk |
| | Mouse (C57B1/6) | H-2Db |
| | Mouse (B10.s) | H-2s |
| 297-318 (ref. 11) | Human | |
| 338-347 (ref. 16) | Human | HLA-B37 |
| 341-362 (ref. 11) | Human | |
| 413-435 (ref. 8) | Mouse (C57B1/6) | H-2Db |

TABLE 3
Influenza A Virus T cell Epitopes of Other Viral Proteins.

| Peptide | Host | T cell type | MHC restriction |
|---|---|---|---|
| PB1 (591-599) (ref. 14) | Human | CTL | HLA-A3 |
| HA (204-212) (ref. 16) | Mouse | CTL | H-2Kd |
| HA (259-266) (ref. 16) | Mouse | CTL | H-2Kd |
| HA (252-271) (ref. 7) | Mouse | CTL | H-2Kk |
| HA (354-362) (ref. 16) | Mouse | CTL | H-2Kk |
| HA (518-526) (ref. 16) | Mouse | CTL | H-2Kk |
| HA (523-545) (ref. 10) | Mouse | CTL | |
| NA (76-84) (ref. 16) | Mouse | CTL | H-2Dd |
| NA (192-201) (ref. 16) | Mouse | CTL | H-2Kd |
| M1 (17-29) (ref. 6) | Human | T helper | HLA-DR1 |
| M1 (56-68) (ref. 4) | Human | CTL | HLA-A2 |
| M1 (58-66) (ref. 12) | Human | CTL | HLA-A2 |
| M1 (128-135) (ref. 15) | Human | CTL | HLA-B35 |
| NS1 (122-130) (ref. 15) | Human | CTL | HLA-A2 |
| NS1 (152-160) (ref. 16) | Mouse | CTL | H-2Kk |

References
(1) McMichael et al., J. Exp. Med. 164: 1397-1406, 1986.
(2) Townsend et al., Cell 44: 959-968, 1986.
(3) Bastin et al., J. Exp. Med. 165: 1508-1523, 1987.
(4) Gotch et al., Nature 326: 881-882, 1987.
(5) Bodmer et al., Cell 52: 253-258, 1988.
(6) Ceppelini et al., Nature 339: 392-394, 1989.
(7) Sweetser et al., Nature 342: 180-182, 1989.
(8) Gao et al., J. Immunol. 143: 3007-3014, 1989.
(9) Rotzschke et al., Nature 348: 252-254, 1990.
(10) Milligan et al., J. Immunol. 145: 3188-3193, 1990.
(11) Brett et al., J. Immunol. 147: 984-991, 1991.
(12) Bednarek et al., J. Immunol. 147: 4047-4053, 1991.
(13) Cerundolo et al., Proc. Roy. Soc. Lond. Series B boil. Sci. 244: 169-177, 1991.
(14) DiBrino et al., J. Immunol. 151: 5930-5935, 1993.
(15) Dong et al., Eur. J. Immunol. 26: 335-339, 1996.
(16) Parker et al., Seminars in Virology 7: 61-73, 1996.

TABLE 4
Extracellular Part of M2 Protein of Human Influenza
A Strains

Virus strain (subtype)

| | |
|---|---|
| A/WS/33 (H1N1) | SLLTEVETPIRNEWGCRCNDSSD¹ |
| A/WSN/33 (H1N1) | SLLTEVETPIRNEWGCRCNDSSD |
| A/NWS/33 (H1N1) | SLLTEVETPIRNEWGCRCNDSSD |
| A/PR/8/34 (H1N1) | SLLTEVETPIRNEWECRCNGSSD² |
| A/Fort Monmouth/1/47 | SLLTEVETPTKNEWGCRCNDSSD³ |
| (H1N1) | |
| A/fort Warren/1/50 (H1N1) | SLLTEVETPIRNEWGCRCNDSSD |
| A/JapanxBellamy/57 (H2N1) | SLLTEVETPIRNEWGCRCNDSSD |
| A/Singapore/1/57 (H2N2) | SLLTEVETPIRNEWGCRCNDSSD |
| A/Leningrad/134/57 (H2N2) | SLLTEVETPIRNEWGCRCNDSSD |
| A/Ann Harbor/6/60 (H2N2) | SLLTEVETPIRNEWGCRCNDSSD |
| A/NT/60/68 (hxNy ?) | SLLTEVETPIRNEWGCRCNDSSD |
| A/Aichi/2/68 (H3N2) | SLLTEVETPIRNEWGCRCNDSSD |
| A/Korea/426/68 (H2N2) | SLLTEVETPIRNEWGCRCNDSSD |
| A/Hong Kong/1/68 (H3N2) | SLLTEVETPIRNEWGCRCNDSSD |
| A/Udorn/72 (H3N2) | SLLTEVETPIRNEWGCRCNDSSD |
| A/Port Chalmers/73 (H3N2) | SLLTEVETPIRNEWGCRCNDSSD |
| A/USSR/90/77 (H1N1) | SLLTEVETPIRNEWGCRCNDSSD |
| A/Bangkok/I/79 | SLLTEVETPIRNEWGCRCNDSSD |
| A/Philippines/2/82MS | SLLTEVETPIRNEWGCRCNGSSD² |
| (H3N2) | |
| A/NY/83 (H3N2) | SLLTEVETPIRNEWGCRCNDSSD |
| A/Memphis/8/88 (H3N2) | SLLTEVETPIRNEWGCRCNDSSD |
| A/Beijing/353/89 (H3N2) | SLLTEVETPIRNEWGCRCNDSSD |
| A/Guangdong/39/89 (H3N2) | SLLTEVETPIRNEWGCRCNDSSD |
| A/Kitalcyushu/159/93 | SLLTEVETPIRNEWGCRCNDSSD |
| (H3N2) | |
| A/Hebei/12/93 (H3N2) | SLLTEVETPIRNEWGCRCNDSSD |
| A/Aichi/69/94 (H3N2) | SLLTEVETPIRNEWECRCNGSSD⁴ |
| A/Saga/447/94 (H3N2) | SLLTEVETPIRNEWECRCNGSSD⁴ |
| A/Sendai/c182/94 (H3N2) | SLLTEVETPIRNEWGCRCNDSSD |
| A/Akita/1/94 (H3N2) | SLLTEVETPIRNEWGCRCNDSSD |
| A/Sendai/c384/94 (H3N2) | SLLTEVETPIRNEWGCRCNDSSD |
| A/Miyagi/29/95 (H3N2) | SLLTEVETPIRNEWGCRCNDSSD |
| A/Charlottesville/31/95 | SLLTEVETPIRNEWGCRCNDSSD |
| A/Akita/1/95 (H3N2) | SLLTEVETPIRNEWECRCNGSSD⁴ |
| A/Shiga/20/95 (H3N2) | SLLTEVETPIRNEWGCRCNDSSD |
| A/Tochigi/44/95 (H3N2) | SLLTEVETPIRNEWECRCNGSSD⁴ |
| A/Hebei/19/95 (H3N2) | SLLTEVETPIRNEWECRCNGSSD⁴ |
| A/Sendai/c373/95 (H3N2) | SLLTEVETPIRNEWECRCNGSSD⁴ |
| A/Niigata/124/95 (H3N2) | SLLTEVETPIRNEWECRCNGSSD⁴ |
| A/Ibaraki/1/95 (H3N2) | SLLTEVETPIRNEWECRCNGSSD⁴ |
| A/Kagoshima/10/95 (H3N2) | SLLTEVETPIRNEWECRCNGSSD⁴ |
| A/Gifu/2/95 (H3N2) | SLLTEVETPIRNEWECRCNGSSD⁴ |
| A/Osaka/c1/95 (H3N2) | SLLTEVETPIRNEWECRCNGSSD⁴ |
| A/Fulcushima/140/96 (H3N2) | SLLTEVETPIRNEWGCRCNDSSD |
| A/Fulcushima/114/96 (H3N2) | SLLTEVETPIRNEWGCRCNDSSD |
| A/Niigata/137/96 (H3N2) | SLLTEVETPIRNEWGCRCNDSSD |
| A/Hong Kong/498/97 (H3N2) | SLLTEVETPIRNEWGCRCNDSSD |
| A/Hong Kong/497/97 (H3N2) | SLLTEVETPIRNEWGCRCNDSSD |
| A/Hong Kong/470/97 (HIN1) | SLLTEVETPIRNEWGCRCNDSSD |
| A/Shiga/25/97 (H3N2) | SLLTEVETPIRNEWGCRCNDSSD |
| A/Hong Kong/427/98 (H1N1) | SLLTEVETPIRNEWECRCNDSSD⁵ |
| A/Hong Kong/1143/99 (H3N2) | SLLTEVETPIRNEWGCRCNDSSD |
| A/Hong Kong/1144/99 (H3N2) | SLLTEVETPIRNEWGCRCNDSSD |
| A/Hong Kong/1180/99 (H3N2) | SLLTEVETPIRNEWGCRCNDSSD |
| A/Hong Kong/1179/99 (H3N2) | SLLTEVETPIRNEWGCRCNDSSD |

¹All sequences in this table correspond to SEQ ID NO: 43, except otherwise indicated
²SEQ ID NO: 44
³SEQ ID NO: 45
⁴SEQ ID NO: 46

TABLE 5
List of examples of pathogens from which epitopes/antigens/
peptides can be derived

```
VIRUSES:
Flaviviridae
    Yellow Fever virus
    Japanese Encephalitis virus
    Dengue virus, types 1, 2, 3 & 4
    West Nile Virus
    Tick Borne Encephalitis virus
    Hepatitis C virus (e.g., genotypes 1a, 1b, 2a, 2b, 2c, 3a, 4a, 4b,
    4c, and 4d)
Papoviridae:
    Papillomavirus
Retroviridae
    Human Immunodeficiency virus, type I
    Human Immunodeficiency virus, type II
    Simian Immunodeficiency virus
    Human T lymphotropic virus, types I & II
Hepnaviridae
    Hepatitis B virus
Picornaviridae
    Hepatitis A virus
    Rhinovirus
    Poliovirus
Herpesviridae:
    Herpes simplex virus, type I
    Herpes simplex virus, type II
    Cytomegalovirus
    Epstein Barr virus
    Varicella-Zoster virus
Togaviridae
    Alphavirus
    Rubella virus
Paramyxoviridae:
    Respiratory syncytial virus
    Parainfluenza virus
    Measles virus
    Mumps virus
Orthomyxoviridae
    Influenza virus
Filoviridae
    Marburg virus
    Ebola virus
Rotoviridae:
    Rotavirus
Coronaviridae
    Coronavirus
Adenoviridae
    Adenovirus
Rhabdoviridae
    Rabiesvirus
BACTERIA:
Enterotoxigenic E. coli
Enteropathogenic E. coli
Campylobacter jejuni
Helicobacter pylori
Salmonella typhi
Vibrio cholerae
Clostridium difficile
Clostridium tetani
Streptococccus pyogenes
Bordetella pertussis
Neisseria meningitides
Neisseria gonorrhoea
Legionella neumophilus
Clamydial spp.
Haemophilus spp.
Shigella spp.
PARASITES:
Plasmodium spp.
Schistosoma spp.
Trypanosoma spp.
Toxoplasma spp.
Cryptosporidia spp.
Pneumocystis spp.
Leishmania spp.
```

TABLE 6
Examples of select antigens from listed viruses

| VIRUS | ANTIGEN |
|---|---|
| Flaviviridae | |
| Yellow Fever virus | Nucleocapsid, M & E glycoproteins |
| Japanese Encephalitis virus | ″ |
| Dengue virus, types 1, 2, 3 & 4 | ″ |
| West Nile Virus | ″ |
| Tick Borne Encephalitis virus | ″ |
| Hepatitis C virus | Nucleocapsid, E1 & E2 |
| | glycoproteins |
| Papoviridae | |
| Papillomavirus | L1 & L2 capsid protein, E6 |
| | & E7 transforming protein (oncopgenes) |
| Retroviridae | |
| Human Immunodeficiency virus, | gag, pol, vif, tat, vpu, env, nef |
| type I | |
| Human Immunodeficiency virus, | ″ |
| type II | |
| Simian Immunodeficiency virus | ″ |
| Human T lymphotropic virus, | gag, pol, env |
| types I & II | |

TABLE 7
Examples of B and T cell epitopes from listed viruses/antigens

| | | | | |
|---|---|---|---|---|
| VIRUS | ANTIGEN | EPITOPE | LOCATION | SEQUENCE (5′-3′) |

Flaviviridae

| | | | | |
|---|---|---|---|---|
| Hepatitis C | Nucleocapsid | CTL | 2-9 | STNPKPQR |
| | | | | (SEQ ID NO: 48) |
| | | | 35-44 | YLLPRRGPRL |
| | | | | (SEQ ID NO: 49) |
| | | | 41-49 | GPRLGVRAT |
| | | | | (SEQ ID NO: 50) |
| | | | 81-100 | YPWPLYGNEGCGWAGWLLS |
| | | | | (SEQ ID NO: 51) |
| | | | 129-144 | GFADLMGYIPLVGAPL |
| | | | | (SEQ ID NO: 52) |
| | | | 132-140 | DLMGYIPLV |
| | | | | (SEQ ID NO: 53) |
| | | | 178-187 | LLALLSCLTV |
| | | | | (SEQ ID NO: 54) |
| | E1 glycoprotein | CTL | 231-250 | REGNASRCWVAVTPTVATRI |
| | | | | (SEQ ID NO: 55) |
| | E2 glycoprotein | CTL | 686-694 | STGLIHLHQ |
| | | | | (SEQ ID NO: 56) |
| | | | 725-734 | LLADARVCSC |
| | | | | (SEQ ID NO: 57 |
| | | | 489-496 | CWHYPPRPCGI |
| | | | | (SEQ ID NO: 5 |
| | | | 569-578 | CVIGGVGNNT |
| | | | | (SEQ ID NO: 59 |
| | | | 460-469 | RRLTDFAQGW |
| | | | | (SEQ ID NO: 6 |
| | | | 621-628 | TINYTIFK |
| | | | | (SEQ ID NO: 61) |
| | | B cell | 384-410 | ETHVTGGNAGRTTAGLVGLL |
| | | | | TPGAKQN |
| | | | | (SEQ ID NO: 62) |
| | | | 411-437 | IQLINTNGSWHINSTALNCNE |
| | | | | SLNTGW |
| | | | | (SEQ ID NO: 63) |
| | | | 441-460 | LFYQHKFNSSGCPERLASCR |
| | | | | (SEQ ID NO: 64) |
| | | | 511-546 | PSPVVVGTTDRSGAPTYSW |
| | | | | GANDTDVFVLNNTRPPL |
| | | | | (SEQ ID NO: 65) |
| | | T helper | 411-416 | IQLINT |
| | | | | (SEQ ID NO: 66) |

Papoviridae

| | | | | |
|---|---|---|---|---|
| HPV 16 | E7 | T helper | 48-54 | DRAHYNI |
| | | | | (SEQ ID NO: 67) |
| | | CTL | 49-57 | RAHYNIVTF |
| | | | | (SEQ ID NO: 68) |
| | | B cell | 10-14 | EYMLD |
| | | | | (SEQ ID NO: 69) |
| | | | 38-41 | IDGP |
| | | | | (SEQ ID NO: 70) |
| | | | 44-48 | QAEPD |
| | | | | (SEQ ID NO: 71) |
| HPV 18 | E7 | T helper | 44-55 | VNHQHLPARRA |
| | | | | (SEQ ID NO: 72) |
| | | | 81-90 | DDLRAFQQLF |
| | | | | (SEQ ID NO: 73) |

The contents of all references cited above are incorporated herein by reference. Use of singular forms herein, such as “a” and “the,” does not exclude indication of the corresponding plural form, unless the context indicates to the contrary.

What is claimed is:

1. A flavivirus vector stably expressing a heterologous sequence inserted at an intergenic site between envelope (E) and non-structural-1 (NS1) proteins of said flavivirus vector.

2. The flavivirus vector of claim 1, wherein said flavivirus vector is a chimeric flavivirus, comprising structural proteins from a first flavivirus and non-structural proteins from a second, different flavivirus.

3. The flavivirus vector of claim 2, wherein said chimeric flavivirus comprises pre-membrane and envelope proteins from said first flavivirus and capsid and non-structural proteins from said second, different flavivirus.

4. The flavivirus vector of claim 2, wherein said first and second flaviviruses are, independently, selected from the group consisting of Japanese encephalitis, Dengue-1, Dengue-2, Dengue-3, Dengue-4, Yellow fever, Murray Valley encephalitis, St. Louis encephalitis, West Nile, Kunjin, Rocio encephalitis, Ilheus, Tick-borne encephalitis, Central European encephalitis, Siberian encephalitis, Russian Spring-Summer encephalitis, Kyasanur Forest Disease, Omsk Hemorrhagic fever, Louping ill, Powassan, Negishi, Absettarov, Hansalova, Apoi, and Hypr viruses.

5. The flavivirus vector of claim 2, wherein said first flavivirus is a Japanese encephalitis virus.

6. The flavivirus vector of claim 2, wherein said flavivirus vector or said second flavivirus is a yellow fever virus.

7. The flavivirus vector of claim 6, wherein said yellow fever virus is YF17D.

8. The flavivirus vector of claim 2, wherein said heterologous sequence comprises an influenza virus M2 or M2e sequence, or an immunogenic fragment or epitope thereof.

9. The flavivirus vector of claim 2, wherein said heterologous sequence comprises a carboxy-terminal anchor-signal sequence.

10. (canceled)

11. The flavivirus vector of claim 2, wherein said heterologous sequence comprises one or more amino-terminal codons added to optimize cleavage.

12. (canceled)

13. The flavivirus vector of claim 2, wherein said heterologous sequence comprises an immunogenic protein, portion thereof, or immunologic epitope thereof, of a viral, bacterial, fungal, or parasitic pathogen, or an oncogenic or allergenic protein.

14. A chimeric flavivirus vector comprising structural proteins from a first flavivirus, non-structural proteins from a second, different flavivirus, and a heterologous sequence inserted at an intergenic site (i) between non-structural-2B (NS2B) and non-structural-3 (NS3) proteins of said chimeric flavivirus vector, or (ii) in the amino-terminal region of the polyprotein of said chimeric flavivirus vector.

15-27. (canceled)

28. A flavivirus vector expressing a heterologous sequence inserted at an intergenic site in the amino terminal region of the flavivirus polyprotein, downstream from the main cyclization signal of the vector.

29-33. (canceled)

34. A flavivirus replicon comprising a non-flavivirus sequence.

35-38. (canceled)

39. A pharmaceutical composition comprising a flavivirus vector of claim 2.

40. A method of delivering a heterologous sequence to a subject, the method comprising administration of a pharmaceutical composition of claim 39 to the subject.

41-43. (canceled)

44. A method of making a flavivirus vector of claim 2, the method comprising introducing a nucleic acid encoding the genome of the flavivirus vector into a cell in which said flavivirus vector replicates, and obtaining the flavivirus vector from the cell or culture supernatant thereof.
